(19) United States
(12) Patent Application Publication
(10) Pub. No.: US 2005/0136439 A1
(43) Pub. Date: Jun. 23, 2005

(54) Novel methods of inorganic compound discovery and synthesis

(75) Inventors: Bruce E. Eaton, Cary, NC (US); Daniel L. Feldheim, Cary, NC (US); Magda Dolska, Raleigh, NC (US); Lina A. Gugliotti, Raleigh, NC (US)

Correspondence Address: MYERS BIGEL SIBLEY & SAJOVEC, PO BOX 37428, RALEIGH, NC, 27627, US

(73) Assignee: North Carolina State University

(21) Appl. No.: 10/937,524

(22) Filed: Sep. 9, 2004

Related U.S. Application Data
(60) Provisional application No. 60/502,394, filed on Sep. 12, 2003.

(51) Int. Cl. C12Q 1/68; C12P 19/34

(52) U.S. Cl. 435/6; 435/91.2

(57) ABSTRACT

The present invention provides methods for the synthesis and/or discovery of inorganic compounds, including organometallic compounds. Also provided are functional nucleic acids for synthesis of inorganic compounds and methods of identifying the same. As another aspect, the invention provides compounds made according to the inventive methods, including palladium plates and cobalt-iron oxides spheres, cubes, fibers and nanotubes.

### RELATED APPLICATION INFORMATION

This application claims the benefit of priority from U.S. provisional patent application Ser. No. 60/502,394, filed Sep. 12, 2003, which is incorporated herein by reference in its entirety.

### FIELD OF THE INVENTION

The present invention relates to methods and reagents for inorganic compound discovery and synthesis; in particular, the present invention relates to methods of using single-stranded nucleic acids for inorganic compound discovery and synthesis.

### BACKGROUND OF THE INVENTION

Controlling the size and shape of metal particles is an important goal of modern colloid science. Catalytic reactivity, magnetic properties, surface-enhanced Raman scattering, and other optical behaviors depend strongly upon metal particle size and shape. While most synthetic procedures result in spherical particles, other geometries such as cubes, rods, and prisms have been synthesized. Such geometric control is often achieved using polymers that adsorb onto certain crystal faces and increase or decrease growth kinetics along those faces. Poly(acrylate), poly(vinylpyrrolidone), and poly(amidoamine) dendrimers are examples of organic macromolecules that have been used successfully to control inorganic crystal structure.

The mechanisms of polymer-directed crystal growth have not been explicated fully. Consequently, despite decades of research, crystal engineering remains largely an empirical discipline. This is due in part to the fact that organic macromolecules are polydisperse in length, and their secondary and tertiary structures are highly dynamic and cannot easily be determined, controlled or varied systematically.

The diverse range of advanced structural, magnetic, and photoresponsive inorganic materials found in nature has motivated the use of peptide and peptidomimetic ligands in materials synthesis and assembly (Cha et al., (2000) Nature 403: 289; Belcher et al., (1996) Nature 381: 56; Hartgerink et al., (2001) Science 294: 1684; Whitling et al., (2000) Adv. Mater. 12: 1377). In contrast to organic macromolecules, large libraries of chemically heterogeneous peptide ligands can be synthesized and screened for their ability to bind certain crystal faces or direct crystal morphologies, thus generating meaningful structure-function relationships. For example, Whittling et al., (2000) Adv. Mater. 12: 1377, synthesized peptide libraries with domains varying by polarity, partition coefficient, globularity, and hydrophobic surface area to define correlations between these descriptors and CdS nanocluster size. Whaley et al., (2000) Nature 405: 665, employed phage display techniques to mine for peptides capable of binding selectively to semiconductor crystal faces. Knowledge of peptide-surface binding affinity was subsequently used to engineer a virus that could bind and organize semiconductor nanocrystals into well-ordered thin film assemblies. Amphiphilic peptides have been used to assemble gold and silica nanoparticles into larger hollow capsules, and to mineralize hydroxyapatite into an architecture similar to natural bone.

In contrast to the fairly extensive work performed to understand peptide-inorganic composites, relatively little research has focused on the interactions between solid-state materials and RNA or DNA. The thermodynamics of dsDNA-CdSe nanocrystal association have been investigated (Mahtab et al., (2000) J. Am. Chem. Soc. 122: 14), and DNA hybridization has been used to assemble network structures of gold nanoparticles (Cao et al., 2001) J. Am. Chem. Soc. 123: 7961; Loweth et al., (1999) Angew. Chem. Int. Ed. Engl. 38: 1808). In addition, several reports have appeared recently which describe metal deposition over dsDNA (Mertig et al., 2002) Nano Lett. 2: 841). However, single-stranded (ss) DNA and RNA, which fold into intricate secondary and tertiary structures, have not previously been used to synthesize inorganic materials.

The present invention addresses a need in the art for improved methods for synthesis and discovery of inorganic compounds.

### SUMMARY OF THE INVENTION

In contrast with the synthetic polymer templates used in prior art methods, RNA and ssDNA are highly structured biopolymers that can reproducibly fold into intricate 3D structures that are conformationally distinct and dictated by their sequence. The inventors have discovered that ss nucleic acid in vitro selection techniques can be adapted to materials synthesis and to isolate nucleotide sequences capable of directing inorganic crystal growth.

The methods of the invention permit the selection and identification of inorganic compounds having a desired property(ies) more readily than with conventional methods. Moreover, the compounds of the invention may have improved homogeneity (e.g., in composition, size distribution, physical and chemical properties, and the like) as compared with compounds produced by traditional processes.

The methods and compositions of the invention have numerous uses; for example, they can be adapted to identify more environmentally friendly aqueous, low temperature routes to materials otherwise synthesized under harsh conditions, and speed the discovery of alloys and intermetallic compounds with desirable catalytic activities or physical properties. Further, the inventive methods can facilitate a better understanding of the mechanism of ss nucleic acid assembly of inorganic solid-state materials and systematic investigation of alloys, intermetallic compounds and material compositions not easily achieved by traditional methods. The invention can also be used to identify new catalytic materials for hydrogen storage, water splitting, direct methanol fuel cells and magnetic devices and sensors. Other applications of the invention include but are not limited to discovery and/or synthesis of materials for photovoltaics, transparent semi-conductors, magnetic semi-conductors, superconductors, field emitters and silicon quantum dots.

Further, ss nucleic acid assembly of nanomaterials can provide major benefits in the synthesis of well-defined particle shapes, compositions and function. Single-stranded nucleic acids can also be used in affinity purification and assembly of specific nanoparticles with desired properties, including the discovery of new catalytic nanoparticles.

Additionally, the evolutionary chemistry (EC) process provided by the present invention provides insight as to what is possible for a range of metal colloids or metal ion compositions as materials for catalysis, and can be used to select for specific particle sizes, shapes, and concomitant catalytic specificities and efficiencies.

Some of the attributes of ss nucleic acid in vitro selection techniques disclosed herein are:
1. A large library of single-stranded nucleic acid sequences (e.g., at least about 10⁸) can be used to select for new compounds not readily synthesized by conventional methods;
2. Multiple metal colloids can be tested simultaneously and selected;
3. If desired, modification of the nucleic acid to include new functional groups (e.g., for catalysis or metal ion binding) can be accomplished. This is a distinguishing feature as compared with in vitro protein evolution techniques;
4. High selectivity can be achieved for specific structures or properties;
5. A complex mixture of metal ions can be used to discover new materials and nanoparticle catalysts.

Accordingly, as a first aspect, the present invention provides a method of producing an inorganic compound product comprising contacting a single-stranded nucleic acid with a metal donor for a time and under conditions sufficient for the production of an inorganic compound product comprising the metal.

As a further aspect, the invention also provides a method of producing an inorganic compound product comprising:
(a) contacting a pool of single-stranded nucleic acids with a metal donor so that an inorganic compound product comprising the metal is assembled;
(b) partitioning nucleic acids that assemble inorganic compound products having a selected property;
(c) generating an enriched pool of single-stranded nucleic acids from the partitioned single-stranded nucleic acids of (b); and
(d) repeating (a) to (c) at least one additional time to produce an inorganic compound product.

In still other embodiments, the invention provides a method of isolating a single-stranded nucleic acid which is able to assemble an inorganic compound product, comprising:
(a) contacting a pool of single-stranded nucleic acids with a metal donor so that an inorganic compound product comprising the metal is assembled;
(b) partitioning nucleic acids that assemble inorganic compounds having a selected property;
(c) generating an enriched pool of single-stranded nucleic acids; and
(d) repeating (a) to (c) at least one additional time to produce an inorganic compound product, thereby isolating a single-stranded nucleic acid which is able to assemble an inorganic compound product.

In particular embodiments of the foregoing methods, the single-stranded nucleic acid is RNA or DNA. In other embodiments the inorganic compound product has a size from about 1 nm to about 20 μm.

As yet another aspect, the invention provides an inorganic solid-state material consisting essentially of a palladium plate (optionally, a magnetic or ferromagnetic palladium plate) and having a size of at least about 50 nanometers.

The invention further provides an inorganic solid-state material consisting essentially of cobalt-iron oxides (e.g., fibers or wires, nanotubes, nanocapsules, spheres and/or cubes).

Also provided are functional RNAs that mediate formation of an inorganic compound.

These and other aspects of the invention are set forth in more detail in the description of the invention below.

### BRIEF DESCRIPTION OF THE DRAWINGS

FIG. 1 depicts the selection scheme for identifying RNAs involved in RNA-mediated crystal growth of Pd metal particles.

FIG. 2A shows transmission electron microscopy (TEM) analysis of the Pd particles of undefined shape produced in 2 hours by the starting random RNA library.

FIG. 2B shows TEM analysis Pd particles created by the evolved RNA cycle 8 pool after 2 hours.

FIG. 2C shows a magnified image of a Pd particle created by the evolved RNA cycle 8 pool after 2 hours.

FIG. 3 shows the distribution of Pd hexagonal particles measured by TEM for the evolved pool and isolate 17 after 2 hours incubation with Pd₂(DBA)₃ (100 μM).

FIG. 4 depicts the scheme for the in vitro selection cycle used to synthesize and identify RNA molecules involved in formation of cobalt-iron oxide compounds.

FIG. 5 illustrates the partitioning steps involved in isolating RNA-cobalt iron oxide magnetic particle clusters. (a) Counterselection—removal of unspecific binding; (b) 12 hour incubation with the magnet; (c) solution and nonmagnetic material removal; (d) four washes with 200 μL of 1× buffer; and (e) resuspension of RNA/cobalt iron oxide magnetic particle clusters in 100 μL of deionized water.

FIG. 6 shows TEM analysis of large (−40 nm) and small (−10 nm) cobalt-iron oxide sphere and cubes containing RNA.

FIG. 7 shows large cobalt iron oxides in the form of fibers present in fractions not retained by the magnet during partitioning.

FIG. 8 shows electron microscope images of cobalt iron oxide nanocapsules (empty spheres) and nanotubes.

FIG. 9. Illustration of in vitro selection of methanol oxidation catalysts. A random pool RNA library nucleates and grows alloy particles. The particles are cast on an electrode surface and methanol oxidation is induced with an applied potential. Active alloys convert methanol into protons causing a local pH change. The acidic environment denatures the RNA bound to the catalytically active particle. The RNA sequences that mediate the formation of the active particle are collected downstream, while the RNA bound to inactive particles remains behind.

FIG. 10. Illustration of a three electrode configuration used in the in vitro selection of methanol oxidation catalysts.

### DETAILED DESCRIPTION OF THE INVENTION

The present invention will now be described with reference to the accompanying drawings, in which preferred embodiments of the invention are shown. This invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein, which do not depart from the instant invention, will be apparent to those skilled in the art in light of the disclosure.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

As used in the description of the invention and the appended claims, the singular forms “a,” “an” and “the” are intended to include the plural forms as well, unless the context clearly indicates otherwise.

All publications, patent applications, patents, and other references mentioned herein are incorporated by reference in their entirety.

The present invention is based, in part, on the novel and unexpected discovery by the inventors that ss nucleic acids can mediate assembly of inorganic compounds, including inorganic compounds having new and desirable characteristics. Thus, the present invention provides new methods and reagents for inorganic compound synthesis and discovery. According to one aspect of the invention, a ss nucleic acid(s) is used to synthesize an inorganic compound. As one embodiment, the present invention provides a method of producing an inorganic compound product comprising contacting a ss nucleic acid with a metal donor for a time and under conditions sufficient for the production of an inorganic compound product comprising the metal.

The invention also encompasses discovery methods for identifying and synthesizing new inorganic compounds. In a representative embodiment, the present invention provides methods of producing an inorganic compound product comprising (a) contacting a pool of ss nucleic acids with a metal donor so that an inorganic compound product comprising the metal is assembled; (b) partitioning nucleic acids that assemble inorganic compound products having a selected property; (c) generating an enriched pool of ss nucleic acids from the partitioned ss nucleic acids of (b); and (d) repeating (a) to (c) at least one additional time to produce an inorganic compound product.

As used herein, the term “inorganic compound product” is intended broadly and encompasses solid-state, colloidal, and soluble (e.g., a soluble catalytic complex) compounds. Also encompassed are crystalline, semi-crystalline and amorphous compounds. In particular embodiments, the inorganic compound is an organometallic compound. In some embodiments, the inorganic compound product is a solid-state material in the form of particles, which can further be in the form of a plate (e.g., having a width less than about 40, 30, 20, 10 or 5 nM or less), fiber or wire, tube, sphere, cube, prism or cluster and the like, all of which can further be crystalline, semi-crystalline or amorphous. As used herein, “particles” include nanoparticles and microparticles, which further include nanospheres, nanotubes, nanocapsules (e.g., hollow spheres), microspheres, microtubes and microcapsules. There are no particular size limits on the inorganic compound product. In representative embodiments of the invention, the inorganic compound product is at least about 0.1 nm, 0.5 nm, 1 nm, 10 nm, 20 nm, 50 nm, 100 nm and/or less than about 200 nm, 300 nm, 500 nm, 1 μm, 5 μm, 10 μm, 20 μm, 50 μm, 100 μm, 500 μm, 1 mm, 2 mm, 5 mm, 10 mm, 20 mm, 50 mm, 100 mm or more in size.

The inorganic compound product can further be two-dimensional or three-dimensional in structure.

In one exemplary embodiment, the inorganic compound product is an nucleic acid/metal cluster (e.g., a soluble RNA/metal cluster). The nucleic acid metal cluster can further be a functional compound that mediates formation of inorganic or organic compounds, e.g., an RNA/metal cluster that mediates the formation of an organic polymer.

Any element in the periodic table as shown in Table 1 can be used to synthesize the inorganic compounds of the invention. For example, in representative embodiments, the invention is practiced to produce pure

TABLE 1
Periodic Table of the Elements

| | Group** | | | | | | | | | | | | | | | | | |
|---|---|---|---|---|---|---|---|---|---|---|---|---|---|---|---|---|---|---|
| Period | 1 | | | | | | | | | | | | | | | | | 18 |
| | IA | | | | | | | | | | | | | | | | | vIIIA |
| | 1A | | | | | | | | | | | | | | | | | 8A |
| | 1 | 2 | | | | | | | | | | | 13 | 14 | 15 | 16 | 17 | 2 |
| 1 | H | IIA | | | | | | | | | | | IIIA | IVA | VA | VIA | VIIA | HE |
| | 1.008 | 2A | | | | | | | | | | | 3A | 4A | 5A | 6A | 7A | 4.003 |
| | 3 | 4 | | | | | | | | | | | 5 | 6 | 7 | 8 | 9 | 10 |
| 2 | Li | Be | | | | | | | | | | | B | C | N | O | F | Ne |
| | 6.941 | 9.012 | | | | | | | | | | | 10.81 | 12.01 | 14.01 | 16.00 | 19.00 | 20.18 |
| | 11 | 12 | 3 | 4 | 5 | 6 | 7 | 8 | 9 | 10 | 11 | 12 | 13 | 14 | 15 | 16 | 17 | 18 |
| 3 | Na | Mg | IIIB | IVB | VB | VIB | VIIB | | VIII | | IB | IIB | Al | Si | P | S | Cl | Ar |
| | 22.99 | 24.31 | 3B | 4B | 5B | 6B | 7B | | — | | 1B | 2B | 26.98 | 28.09 | 30.97 | 32.07 | 35.45 | 39.95 |
| | | | | | | | | | 8 | | | | | | | | | |
| | 19 | 20 | 21 | 22 | 23 | 24 | 25 | 26 | 27 | 28 | 29 | 30 | 31 | 32 | 33 | 34 | 35 | 36 |
| 4 | K | Ca | Sc | Ti | V | Cr | Mn | Fe | Co | Ni | Cu | Zn | Ga | Ge | As | Se | Br | Kr |
| | 39.10 | 40.08 | 44.96 | 47.88 | 50.94 | 52.00 | 54.94 | 55.85 | 58.47 | 58.69 | 63.55 | 65.39 | 69.72 | 72.59 | 74.92 | 78.96 | 79.90 | 83.80 |
| | 37 | 38 | 39 | 40 | 41 | 42 | 43 | 44 | 45 | 46 | 47 | 48 | 49 | 50 | 51 | 52 | 53 | 54 |
| 5 | Rb | Sr | Y | Zr | Nb | Mo | Tc | Ru | Rh | Pd | Ag | Cd | In | Sn | Sb | Te | I | Xe |
| | 85.47 | 87.62 | 88.91 | 91.22 | 92.91 | 95.94 | (98) | 101.1 | 102.9 | 106.4 | 107.9 | 112.4 | 114.8 | 118.7 | 121.8 | 127.6 | 126.9 | 131.3 |
| | 55 | 56 | 57 | 72 | 73 | 74 | 75 | 76 | 77 | 78 | 79 | 80 | 81 | 82 | 83 | 84 | 85 | 86 |
| 6 | Cs | Ba | La* | Hf | Ta | W | Re | Os | Ir | Pt | Au | Hg | Tl | Pb | Bi | Po | At | Rn |
| | 132.9 | 137.3 | 138.9 | 178.5 | 180.9 | 183.9 | 186.2 | 190.2 | 190.2 | 195.1 | 197.0 | 200.5 | 204.4 | 207.2 | 209.0 | (210) | (210) | (222) |
| | 87 | 88 | 89 | 104 | 105 | 106 | 107 | 108 | 109 | 110 | 111 | 112 | | 114 | | 116 | | 118 |
| 7 | Fr | Ra | Ac˜ | Rf | Db | Sg | Bh | Hs | Mt | — | — | — | | — | | — | | — |
| | (223) | (226) | (227) | (257) | (260) | (263) | (262) | (265) | (266) | ( ) | ( ) | ( ) | | ( ) | | ( ) | | ( ) |

| | | | | | | | | | | | | | | |
|---|---|---|---|---|---|---|---|---|---|---|---|---|---|---|
| Lanthanide | 58 | 59 | 60 | 61 | 62 | 63 | 64 | 65 | 66 | 67 | 68 | 69 | 70 | 71 |
| Series* | Ce | Pr | Nd | Pm | Sm | Eu | Gd | Tb | Dy | Ho | Er | Tm | Yb | Lu |
| | 140.1 | 140.9 | 144.2 | (147) | 150.4 | 152.0 | 157.3 | 158.9 | 162.5 | 164.9 | 167.3 | 168.9 | 173.0 | 175.0 |
| | 90 | 91 | 92 | 93 | 94 | 95 | 96 | 97 | 98 | 99 | 100 | 101 | 102 | 103 |
| Actinide Series˜ | Th | Pa | U | Np | Pu | Am | Cm | Bk | Cf | Es | Fm | Md | No | Lr |
| | 232.0 | (231) | (238) | (237) | (242) | (243) | (247) | (247) | (249) | (254) | (253) | (256) | (254) | (257) |

carbon compounds. In other embodiments, the inorganic compound is an organometallic compound. The terms “metal” and “organometallic” are intended broadly and generally include the elements in the periodic table other than the noble gases (helium, neon, argon, krypton, xenon, radon), halogens (fluorine, chlorine, bromine, iodine and astatine) and other and non-metals such as hydrogen, oxygen, carbon, sulfur, nitrogen, phosphorus and selenium. Any metal, or alloy thereof, known in the art can be used according to the present invention. Suitable metals include, but are not limited to transition metals, metalloids, alkali metals, alkaline earth metals, elements in the Lanthanide series, elements in the Actinide series, other metals such as aluminum, gallium, indium, tin, thallium, lead and bismuth. Particular metallic elements of interest include but are not limited to palladium, cobalt, platinum, aluminum, iron, rughenium, silver, gold, tin, lead, cadmium, copper, nickel and silicon and metals useful in forming semi-conductors.

The transition metals generally include scandium, titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, gold, mercury, rutherfordium, dubnium, seaborgium, bohrium, hassium and meitnerium.

Metalloids generally include boron, silicon, germanium, arsenic, antimony, tellurium and polonium.

Alkali metals generally include lithium, sodium, potassium, rubidium, cesium and francium.

Alkaline earth metals generally include beryllium, magnesium, calcium, strontium, barium and radium.

The Lanthanide series generally includes lanthanum, cerium, praseodymium, neodymium, promethium, samarium, europium, gadolinium, terbium, dysprosium, holmium, erbium, thulium, ytterbium and lutetium.

The Actinide series generally includes actinium, thorium, protactinium, uranium, neptunium, plutonium, americium, curium, berkelium, californium, einsteinium, fermium, mendelevium, nobelium and lawrencium.

The term “metal” further encompasses any newly-discovered or characterized metallic element.

Those skilled in the art will appreciate that the inorganic compound products of the invention can further comprise organic elements including carbon, oxygen, hydrogen, nitrogen, phosphorus and/or sulfate, and the like.

As used herein an “alloy” is a homogeneous mixture or solid solution of two or more metals or metallic and non-metallic elements. An alloy can be binary, ternary, quaternary, etc., depending on the number of metals used to form the alloy.

An “intermetallic compound” is similar to an alloy, but differs in that an alloy is typically a disordered solid solution of two or more metallic elements or metallic and non-metallic elements, which does not have any particular chemical formula and is typically best described as a base material to which certain percentages of other elements have been added. An example is type 304, a grade of stainless steel, which has the composition Fe-18% CrO8% Ni. An intermetallic compound, on the other hand, is a particular chemical compound based on a definite atomic formula, with a fixed or narrow range of chemical composition. An example is the nickel aluminide Ni₃Al. Conventional alloys are linked with relatively weak metallic bonds, whereas the bonds in intermetallics may be partly ionic or covalent. Alternatively, the bonding in an intermetallic may be entirely metallic, but the atoms of the individual elements are “ordered” in that they take up preferred positions within the crystal lattice.

In particular embodiments of the invention, the inorganic compound product comprises, consists essentially of, or consists of a metal or metal alloy or intermetallic compound. In other representative embodiments, the inorganic compound product comprises, consists essentially of, or consists of a crystalline, semi-crystalline or amorphous solid-state material.

The metal donor can be any molecule, organic or inorganic, that provides the metal(s) substrate to the synthetic reaction as known by those skilled in the art. Generally, it is desirable that the metal is not so strongly bonded to the precursor that the metal is not efficiently transferred from the precursor in the course of the synthetic reaction. Further, the metal donor can be chosen to provide the metal(s) in the correct valency for the synthetic reaction (i.e., to prevent the need for additional oxidation or reduction reactions) or to otherwise provide the metal in a suitable form. The metal donor can provide more than one metal or, alternatively, more than one metal donor can be used in the synthesis reaction.

In particular embodiments, the reaction mixture further comprises a reducing agent such as H₂ or NaBH₄ or, alternatively, O₂ to form metal oxide compounds.

“Single stranded nucleic acids” include ssRNA, ssDNA and chimeras thereof as well as chemically modified forms (see below). Those skilled in the art will understand that a ss nucleic acid as defined herein may form hairpin structures by intramolecular base-pairing under certain conditions.

A “pool” of ss nucleic acids is a library or any other composition or mixture containing a plurality of distinct nucleic acid sequences. The pool can be derived from natural sources (e.g., from a library derived from a particular organism, cell and the like) or can be partially or completely synthetic. Further, the sequences of the individual nucleic acids in the pool can be completely variable or, alternatively, can contain both fixed and variable sequences. For example, fixed regions can be included in the nucleic acids that comprise recognition sites for enzymes (e.g., promoters), restriction sites, and the like. The initial pool of nucleic acids used for the selection protocols of the invention can contain any convenient and suitable number of unique nucleic acid sequences to achieve the desired result, for example, at least about 10⁸, 10⁹, 10¹⁰, 10¹¹, 10¹², 10¹³ or 10¹⁴ theoretically unique sequences. In illustrative embodiments, the pool theoretically contains from about 10⁶, 10⁷, 10⁸, 10⁹ or 10¹⁰ unique sequences and/or up to about 10¹¹, 10¹², 10¹³, 10¹⁴, 10¹⁵, 10¹⁶ or 10¹⁷ unique sequences. The ss nucleic acids within the pool can be of any suitable length to give the desired number of unique sequences (e.g., a variable region of N bases in length will theoretically give rise to 4^{N} unique sequences). Typically, the variable region is from about 10 to about 75, about 20 to about 60, about 25 to about 50, or about 35 to about 45 bases long, and the entire length of the ss nucleic acid is at lest about 40, 50, 75 or 100 bases and/or less than about 125, 150, 200 or 250 bases in length. Those skilled in the art will understand that it is advantageous to choose a variable region that gives the appropriate level of sequence diversity; however, long sequences can be expensive to synthesize and more difficult to manipulate (e.g., to amplify).

The invention can be practiced to identify a “functional” ss nucleic acid(s) that mediates (i.e., facilitates) assembly of an inorganic compound product, optionally having a desired characteristic(s). Without being limited to any particular mechanism of action, the ss nucleic acid can act as a true catalyst (i.e., increases the reaction rate without being consumed in the reaction), an activator (i.e., increases the reaction rate and is consumed in the reaction), a template, scaffold or seed (e.g., for nucleation) to facilitate synthesis of the inorganic compound, a cofactor in the synthesis reaction and/or can mediate assembly of the compound by any other mechanism. Further, more than one ss nucleic acid may act in concert, concurrently or sequentially, to assemble the inorganic compound.

One advantage of the methods of the invention is that a wide range of functionality can be achieved in the ss nucleic acid molecule by using modified nucleobases. For example, thiol and pyridyl groups have been reported to demonstrate enhanced metal binding. In particular embodiments, the modified base is a thiol-modified uracil, a fluoro-modified uracil, a methoxy-modified uracil, an azido-modified uracil, an imidazole-modified uracil, a pyridyl-modified uracil, pyridylmethyl-modified uracil, an oxime-modified uracil, a carboxylate-modified uracil, an amine-modified uracil, a phosphine-modified uracil and/or a phosphite-modified uracil. Typically, but not necessarily, the modified nucleobase is completely substituted for the naturally-occurring base when synthesizing the pool of ss nucleic acids. Numerous modified nucleobases for incorporation into RNA or DNA are known in the art, see e.g., Tarasow et al., 1998 Biopoly 48: 29; Chellisserylkattil & Ellington, (2004) Nature Biotech. 22: 1155-1160; U.S. Pat. No. 5,959,100; U.S. Pat. No. 5,945,527; U.S. Pat. No. 5,719,273; U.S. Pat. No. 5,783,679; U.S. Pat. No. 5,633,361; U.S. Pat. No. 5,591,843; U.S. Pat. No. 5,428,149; U.S. Pat. No. 6,300,074; U.S. Pat. No. 5,962,219; U.S. Pat. No. 5,998,142; U.S. Pat. No. 5,858,600; U.S. Pat. No. 5,789,160; U.S. Pat. No. 5,723,592; U.S. Pat. No. 5,723,289; U.S. Pat. No. 5,773,598; U.S. Pat. No. 6,030,776; U.S. Pat. No. 6,048,698; U.S. Pat. No. 6,048,698; U.S. Pat. No. 5,763,595; U.S. Pat. No. 5,705,337; U.S. Pat. No. 5,637,459; U.S. Patent Application publication 20030099945; the disclosures of which are incorporated herein by reference in their entireties.

Additionally, diverse functionality can be achieved in the ss nucleic acid molecule by using modified ribose monomers. For example, amino and fluoro groups are widely known to enhance RNA stability. Numerous modified riboses for incorporation into RNA are known in the art. In particular embodiments, the modified ribose is a thiol-modified ribose, a methoxy-modified ribose, an oxime-modified ribose, an azido-modified ribose, a fluoro-modified ribose, a carboxylate-modified ribose, an amine-modified ribose, an imidazole-modified ribose, a pyridyl-modified ribose, a pyridylmethyl-modified ribose, a phosphine-modified ribose, and/or a phosphite-modified ribose. Typically, but not necessarily, the modified ribose is completely substituted for the naturally-occurring base when synthesizing the pool of ss nucleic acids.

In one representative embodiment, the modified ribose monomer is a 2′-modified ribose monomer. 2′-modified riboses are known in the art (see, e.g., Chellisserylkattil & Ellington, (2004) Nature Biotech. 22: 1155-1160), and include but are not limited to a 2′-position thiol-modified ribose, a 2′-methoxy ribose, a 2′-oxime-modified ribose, a 2′-azido-modified ribose, a 2′ fluoro-modified ribose, a 2′-carboxylate-modified ribose, a 2′-amine-modified ribose, a 2′-phosphine-modified ribose, and/or a 2′-phosphite-modified ribose. Exemplary 2′-modified bases include but are not limited to 2′-fluoro-CTP, 2′-fluoro-UTP, 2′-fluoro-GTP, 2′-fluoro-ATP, 2′-amino-CTP, 2-amino-UTP, 2′-amino-GTP, 2′-amino-ATP, 2′-O-methyl CTP, 2′-O-methyl UTP, 2′-O-methyl GTP, 2′-O-methyl ATP, 2′-azido-CTP, 2′-azido-UTP, 2′-azido-GTP and/or 2′-azido-ATP.

As a further illustration, Table 2 contains a nonexhaustive list of functional groups that can be used to produce modified nucleobases for incorporation into RNA or DNA. The asterisks indicate the point of attachment to the nucleobase, but does not limit the chain length to that shown in the table. In representative embodiments, the point of attachment for pyrimidine bases (cytidine, thymidine, uridine) is at the 7-position and for purine bases (guanosine, adenosine) is at the 5-position. In addition, modifications of DNA by incorporation of functional groups into the triphosphates region is also known in the art.

TABLE 2

| | |
|---|---|
| 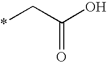 | 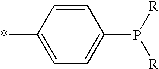 |
| 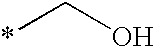 | 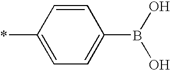 |
| 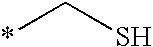 | 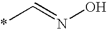 |
| 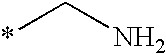 | 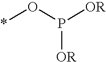 |
| 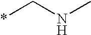 | 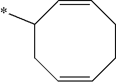 |
| 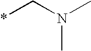 | 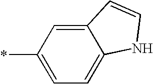 |
| 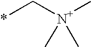 | 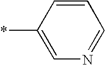 |
| 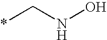 | 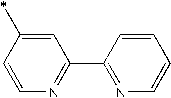 |
| 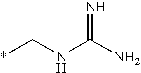 | 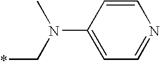 |
| 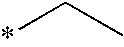 | 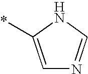 |
| 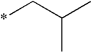 | 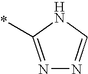 |
| 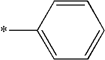 | 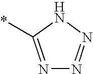 |
| 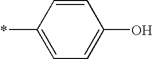 | 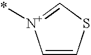 |
| 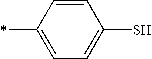 | 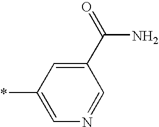 |
| 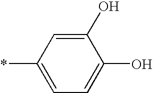 | 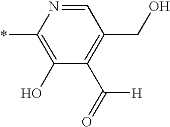 |

*Asterisks indicate point of attachment but do not limit chain length to that shown in the table.

Table 3 shows exemplary 5-position modified uridines that can be used in the methods of the present invention. In illustrative methods, the modified uridine is a thiol-modified uracil, a fluoro-modified uracil, a methoxy-modified uracil, an azido-modified uracil, an imidazole-modified uracil, a pyridyl-modified uracil, pyridylmethyl-modified uracil, an oxime-modified uracil, a carboxylate-modified uracil, an amine-modified uracil, a phosphine-modified uracil and/or a phosphite-modified uracil.

TABLE 3

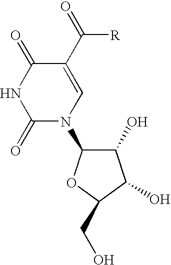

R

| | |
|---|---|
| 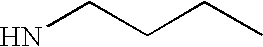 | 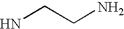 |
| 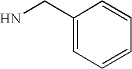 | 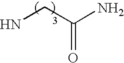 |
| 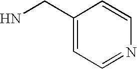 | 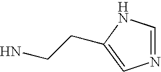 |
| 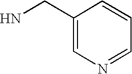 | 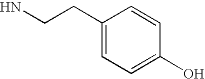 |
| 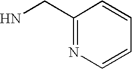 | 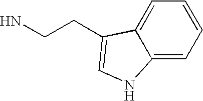 |

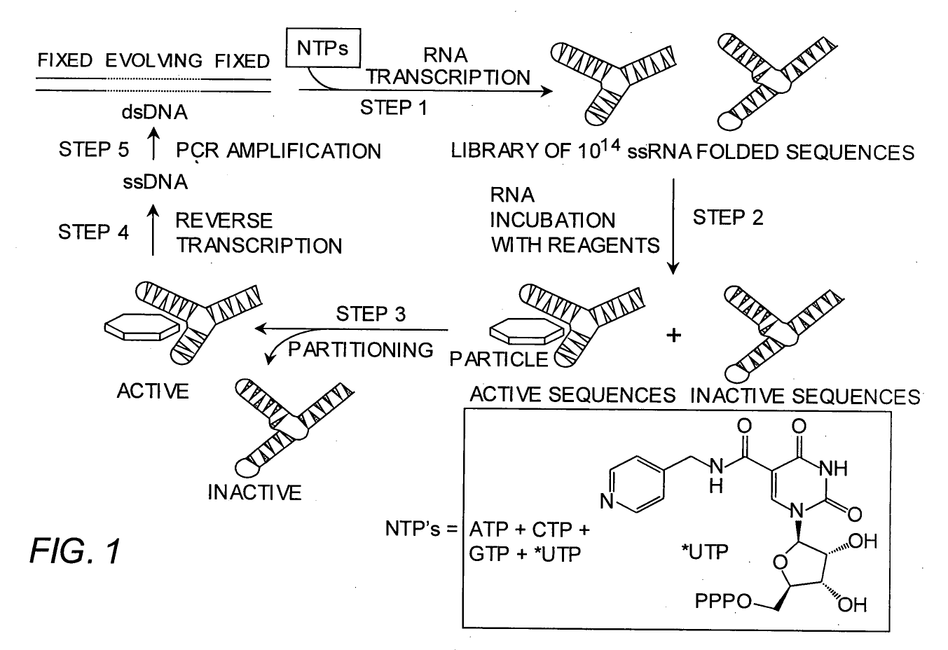

In representative embodiments, the invention provides a combinatorial discovery approach that generally employs an initial pool of unique ss stranded nucleic acid sequences (see FIG. 1 below). In the particular embodiment shown in FIG. 1, a ss RNA pool is exposed to a solution containing an organic or inorganic metal precursor(s). Upon reducing the metal precursor, particles nucleate and grow on the functional RNA. However, because each RNA sequence in the initial mixture differs in primary sequence and secondary structure, many different inorganic crystal types result. In fact, one expects that some of the initial RNA sequences are incapable of nucleating a crystal, others may grow crystals differing in size (due to slow nucleation), shape (due to slow growth on one crystal face), or physical property(ies) (e.g., magnetism). A separation is then performed to isolate the desired structure. For example, in a first step or “selection cycle 1,” RNA sequences not bound to a crystal can be removed by centrifugation. These sequences are “partitioned” or “selected” out; that is, only those sequences that grow crystals survive and are carried forward to the second cycle. The RNA that is carried forward may constitute a minor fraction of the overall sample. However, the “winning” RNAs can be reverse transcribed into cDNA, amplified using DNA amplification techniques (e.g., polymerase chain reaction; PCR), and converted back into RNA for the next cycle. In subsequent cycles, more stringent and/or different selection pressures can be imposed. Rather than simply selecting for RNA that grows a crystal, one can select for RNA that grows crystals possessing a certain catalytic, electronic, photophysical and/or magnetic property and the like. After several cycles (e.g., around 10), the initial RNA pool of sequences is narrowed to a much smaller pool (e.g., tens to hundreds) containing families of sequences that grow crystals with the desired property. Remarkably, if 1 RNA sequence in 1 billion grows the desired crystal, it can be isolated, amplified, and recovered in pure form from an initial mixture of crystals produced by a library of 10¹⁴ molecules.

Mirkin, “Programming the Assembly of Two- and Three-Dimensional Architectures with DNA and Nanoscale Inorganic Building Blocks,” Inorg. Chem. 39: 2258 (2000), describes a method of assembling nanoscale inorganic building blocks into macroscopic materials using matched sets of double-stranded (ds)DNA molecules with “hanging” single-stranded ends as interconnector molecules (see also, U.S. Pat. Nos. 6,582,921; 6,506,564; and 6,417,340; all to Mirkin et al.). This method can be readily distinguished from the present invention. The method of Mirkin requires pre-existing knowledge of the nucleotide sequences and a predefined end-product, and further requires sequence-specific hybridization between complementary DNA sequences. This method organizes known nanometer sized compounds into aggregates. In addition, the method of Mirkin relies on the limited two-dimensional rigid structure of the DNA double helix to act as an effective interconnector molecule. In contrast, the present invention can be practiced to identify new inorganic compounds as well as single-stranded nucleic acids that mediate formation thereof without pre-existing information regarding the sequence or compound composition. Further, the present invention generally does not require complementary base-pairing between nucleic acids, although basepairing and cooperativity among molecules can occur in some instances. Finally, the invention described herein uses ss nucleic acids, which have a flexibility that permits formation of a diversity of conformationally distinct two- and three-dimensional structures. Such structures are capable of increased functionality as compared with the relatively inert connector molecules described by Mirkin.

The contacting step between the metal donor and the pool of ss nucleic acids is typically carried out in liquid-phase, although solid-phase and gas-phase can also be used. In particular embodiments, the ss nucleic acids are dispersed on a solid surface. The conditions are generally chosen to avoid denaturing of the ss nucleic acids; such conditions are known by those skilled in the art. The contacting step can be carried out in an aqueous, nonaqueous (e.g., acetonitrile, carbon disulfide, tetrahydro-furan) or water/organic solvent mixtures.

In particular embodiments, the synthetic reaction is carried out at a temperature from just above the freezing temperature of water up to about 40, 45 or 50° C. or even higher. Methods of selecting or designing nucleic acids for stability at high temperatures are known in the art. In particular embodiments, the synthesis is carried out at ambient temperature.

In conventional methods of using synthetic polymers to direct crystal type and size, the concentration of polymer is typically in excess of inorganic precursor, the metal precursor is present at relatively high concentration, and the reaction is performed at elevated temperature. In contrast, in embodiments of the invention, the methods of the present invention can be carried out using much lower concentrations of ss nucleic acid and metal precursor (e.g., by several orders of magnitude) than in previous methods, optionally at ambient temperature. For example, it particular embodiments, the metal precursor is present at a concentration of at least about 100 nM, 500 nM, 1 μM, 5 μM, 10 μM, 50 μM or 100 μM and/or less than about 10 mM, 50 mM, 100 mM, 500 mM, 1 M, 5 M, 10 M, 50 M, 100 M or higher. Alternatively, or additionally, in embodiments of the invention, the ss nucleic acid (as initial RNA pool, enriched RNA pool or purified functional sequences) is present at a concentration of at least about 1 nM, 5 nM, 10 nM, 50 nM, 100 nM or 500 nM and/or less than about 100 μM, 500 μM, 1 mM, 10 mM, 50 mM, 100 mM, 500 mM or 1 M or higher.

A portion of the ss nucleic acids within the pool will mediate the formation of inorganic compounds (as discussed above), and a diverse array of compounds is typically produced. Selection pressure is applied to identify those inorganic compounds, and nucleic acids, that have a characteristic(s) of interest.

The functional ss nucleic acids are then separated or partitioned from the other ss nucleic acids based on a selection criterion (or criteria). Any desired criterion/property can be used to partition the nucleic acids, including but not limited to size, a magnetic property (e.g., magnetism), shape (e.g., a two-dimensional plate, sphere, cube, elongated fiber or a tube), an optical property, luminescence, fluorescence, an electronic property, a photophysical property, crystal structure, or a catalytic property (e.g., methanol oxidation, polymer formation). Selection can additionally be based on speed of the assembly process, e.g., nucleic acids can be partitioned based on those that assemble an inorganic compound product have a desired property within a specified time period (e.g., less than about 5 minutes, 10 minutes, 15 minutes, 30 minutes, 45 minutes, 60 minutes, 90 minutes, 120 minutes and the like).

In embodiments of the invention, the selection pressure is increased over iterations of the cycle (e.g., an increasing magnetic force is applied, selection for larger particles, selection for tighter binding between the nucleic acid and inorganic compound, etc.). In other embodiments, more than one selection criterion is applied concurrently or sequentially. For example, in earlier iterations of the selection cycle, selection can be on the basis of particle size or shape and during later iterations it can be on the basis of another property such as conductance, a magnetic property and the like.

The selection criterion (or criteria) can be applied using any method known in the art for distinguishing compounds on the basis of particular properties. For example, size and shape can be selected using standard techniques involving filtration, sedimentation, centrifugation, chromatography and electrophoreses (e.g., SDS— or nondenaturing PAGE). Movement through a liquid or gel toward a magnet can also be used to partition compounds based on size and/or shape.

A magnet can be used to partition the inorganic compounds based on magnetic properties (e.g., ferromagnetism or paramagnetism).

Catalytic properties can be selected based on the characteristics of the end product of the catalytic reaction. For example, in the case of methanol oxidation, oxidation of methanol to hydrogen and carbon dioxide results in a reduction in local pH, which can be selected for (e.g., by desorption of the ss nucleic acid to a surface, as described below).

As another illustrative example, selection based on luminescent, fluorescent or photophysical properties can be carried out by attaching biotin to the ss nucleic acid using a photocleavable linker. The photocleavable linker is designed to cleave when irradiated with light energy emitted by the most active particles. In a typical selection, the ss nucleic acid library is used to synthesize a range of potential luminescent materials. Each ss nucleic acid contains a biotin at one end connected by the photocleavable linker. The resulting particles are spread out on a surface and excited with light. The particles that emit light will cleave the biotin from the nucleic acid template. Particles that do not emit light (or only low levels of light) will contain an intact biotin-nucleic acid template. The nucleic acids are then denatured from the particles and sent through a streptavidin column. Single-stranded nucleic acid from the most active particles will wash through the column because it is no longer linked to biotin. Single-stranded nucleic acid from inactive particles is retained on the column due to the presence of the biotin molecules.

As an alternative approach, selection based on luminescent, fluorescent or photophysical properties can be carried out by separating the particles using chromatography, and the particles with the desired property are then collected by a sorting device that detects the desired property (e.g., as in fluorescent activated cell sorting).

Generally, at some point in the in vitro synthesis scheme, the functional ss nucleic acid is bound (covalently or non-covalently) to the inorganic compound product. The ss nucleic acid can be directly or indirectly (e.g., by binding to another ss nucleic acid that is directly bound to the inorganic compound) bound to the inorganic compound product. In this manner, by partitioning the compounds of interest, the functional ss nucleic acid that mediates formation of these compounds will typically also be partitioned and, thus, can be input into the next selection cycle. As another example, as described below, in a methanol fuel cell, the most active ss nucleic acids desorb to the electrode and can be partitioned on that basis. As a further non-limiting example, the inorganic compound product can be a soluble nucleic acid-metal complex that mediates organic polymer formation. The soluble nucleic acid-metal complex is associated with the polymer and can be disassociated or cleaved from the polymer to recover the ss nucleic acid, optionally for input into the next round of selection.

Alternatively, functional ss nucleic acids can be isolated and/or identified that do not remain bound to the inorganic compound product by limiting the diffusion of the ss nucleic acid away from the inorganic compound. For example, a gel or viscous liquid can be used to reduce the diffusion of the ss nucleic acid away from the inorganic compound, and the ss nucleic acid can be isolated and/or identified, therefrom.

In another illustrative embodiment, the ss nucleic acid can be dispersed on and/or affixed to a solid support and the inorganic compound (e.g., particles) can be grown on the surface of the solid support. Any solid support known in the art can be used, including but not limited to, plastic plates (including multi-well plates), slides, beads, tubes; glass plates, slides, beads, tubes; chromatography matrices, silica beads, metal beads or other metal surfaces, paper and synthetic membranes, an electrode, and the like. In particular embodiments, the ss nucleic acid is arrayed on a glass or plastic surface (e.g., on a slide or in a multi-well plate). Methods of making nucleic acid arrays are well-known in the art. Physical separation of ss nucleic acids can advantageously facilitate the selection/partitioning process; for example, structural characteristics of the arrayed compound products can be assessed, e.g., using transmission electron microscopy or catalytic activity can be assessed using standard assays (e.g., in a microtiter plate). Further, arrays of compounds are suited to high throughput methods, which can be partially or entirely automated.

Once the functional and inactive ss nucleic acids have been partitioned based on the selection criterion or criteria, a new pool of ss nucleic acids that is enriched for the functional ss nucleic acid sequences is generated. Generation of the enriched pool can be carried out using standard molecular biology techniques (see, e.g., SAMBROOK et al., MOLECULAR CLONING: A LABORATORY MANUAL 2nd Ed. (Cold Spring Harbor, N.Y., 1989); F. M. AUSUBEL et al. CURRENT PROTOCOLS IN MOLECULAR BIOLOGY (Green Publishing Associates, Inc. and John Wiley & Sons, Inc., New York)).

In representative embodiments, the step of generating an enriched pool of ss nucleic acids includes a nucleic acid amplification step. Methods for amplifying nucleic acids are known in the art. Such methods include but are not limited to Polymerase Chain Reaction (PCR; described in U.S. Pat. Nos. 4,683,195; 4,683,202; 4,800,159; and 4,965,188), Strand Displacement Amplification (SDA; described by G. Walker et al., Proc. Nat. Acad. Sci. USA 89, 392 (1992); G. Walker et al., Nucl. Acids Res. 20, 1691 (1992); U.S. Pat. No. 5,270,184), thermophilic Strand Displacement Amplification (tSDA; EP 0 684 315 to Frasier et al.), Self-Sustained Sequence Replication (3SR; J. C. Guatelli et al., Proc Natl. Acad. Sci. USA 87, 1874-78 (1990)), Nucleic Acid Sequence-Based Amplification (NASBA; U.S. Pat. No. 5,130,238 to Cangene), the Qβ replicase system (P. Lizardi et al., BioTechnology 6, 1197 (1988)), or transcription based amplification (D. Y. Kwoh et al., Proc. Natl. Acad. Sci. USA 86, 1173-77 (1989)).

Any technique in the art can be used to manipulate and, optionally, amplify the partitioned ss nucleic acids to generate the enriched pool. Such techniques are well-known and standard in the art. To illustrate, in the case of ssRNA, the partitioned nucleic acids can be used as a template for ssDNA synthesis by reverse transcriptase. dsDNA can then be synthesized by standard techniques, e.g., PCR can be used to amplify the functional sequences and to generate an enriched pool of amplified dsDNA sequences. ssDNA or ssRNA can be readily produced from the dsDNA using standard techniques known to those skilled in the art to produce the enriched pool for the next round of selection.

If selection is based on a pool of ssDNA molecules, typically any amplification technique (e.g., PCR) can be used to amplify the selected ssDNA and to produce dsDNA therefrom. To remove the antisense strand from the sense strand to undergo selection, various modified primers can be used to allow removal of the antisense strand from the PCR pool. Examples include biotinylated primers that allow the removal of ssDNA from dsDNA by heating the dsDNA and capture of the antisense ssDNA on a streptavidin bead, or reactive primers such as thiols that can be chemically trapped on beads or surfaces. The ssDNA is then subject to the selection conditions and the ssDNA isolated from the partitioning step is subject to PCR amplification with modified primers such that the antisense can be removed and the selection cycle repeated.

The steps of contacting the ss nucleic acid with the metal donor, partitioning the functional ss nucleic acids and generating an enriched pool of ss nucleic acids from the partitioned nucleic acids to prime another cycle can be repeated one or more times, typically until an inorganic compound product(s) having a desirable characteristic is produced. The cycle can be repeated at least two, three, four, five, eight, ten, twelve, fifteen, twenty or more times. In particular embodiments, the cycle is repeated from about five to fifteen times or from about seven to twelve times.

Typically, after five or more rounds, the number of unique ss nucleic acids in the pool is reduced (e.g., to hundreds or even tens). Additionally, some of the sequences in the enriched pool can be related, e.g., share conserved sequences or form similar two-dimensional or three-dimensional structures. Once identified, one or more of the functional ss nucleic acid sequences can be synthesized and used to assemble the inorganic compound product without the need for the selection process. Alternatively, in some embodiments, the identified sequences can be modified (e.g., shortened, lengthened and/or altered by deletions, insertions, addition or removal of a modified base, by nucleotide substitutions, addition of other functional sequences to facilitate detection or purification, and the like) for use in synthetic reactions. As another alternative, the sequence and structural information obtained from the final pool of enriched ss nucleic acids can be used to synthesize other ss nucleic acids having similar or enhanced properties.

Accordingly, as another aspect, the present invention provides isolated functional ss nucleic acids that can mediate formation of an inorganic compound product. Single-stranded nucleic acids are as described above. In particular embodiments, the functional ss nucleic acid is identified using a screening method of the invention (described in more detail below). Exemplary functional ss nucleic acids comprise, consist essentially of, or consist of the ss nucleic acids as shown in Tables 1 and 2 below (ie., SEQ ID NOs:1-73) or a functional portion of at least 5, 8, 10, 15 or 20 consecutive nucleotide bases thereof. Other illustrative functional ss nucleic acids comprise, consist essentially of, or consist of one or more of the conserved motifs shown in Tables 1 and 2 below (e.g., as shown by underlining or capitals in Tables 1 and 2 or the patterns indicated in Table 2). Other ss nucleic acids comprise, consist essentially of, or consist of the consensus motifs:

(SEQ ID NO:74)

CYCUUYCUAUYYYCAAWGUMCCAACWAAAAAUGUAYBCCX₁
(wherein X₁ is absent or C);

(SEQ ID NO:75)

CUCCUUAAUACCUYWWAAUACCCCAUCUUUX₁YGWX₁CGUUA
(wherein X₁ is absent or A);

(SEQ ID NO:76)

CUCUUUAUUUCCUUWAWAX₁UACCMMMUCUUAWUGWAUCX₁CC
(wherein X₁ is absent or G);

(SEQ ID NO:77)

MYWMYHWATRHRSTHHAATAAAAWYWMWWACWAWA;
or

(SEQ ID NO:78)

HHYATTWACABNMHSWWMYT;
where
B = C, G, T
D = A, G, T
H = A, C, T
V = A, C, G
R = A or G
Y = C or T
K = G or C
M = A or C
S = G or C
W = A or T

Also encompassed are ss nucleic acids that are variants of the ss nucleic acids described above. Exemplary variants include ss nucleic acids having 1, 2, 3, 4, 5, 6, 7, 8, 9 or 10 substitutions, insertions and/or deletions of nucleotide bases as compared with the ss nucleic acids sequences and motifs described above and/or comprise or lack one or more modified bases (e.g., an imidazole-modified uracil in place of uracil or vice versa) as compared with the ss nucleic acids described above.

The invention further provides methods of isolating a ss nucleic acid which is able to assemble an inorganic compound product, comprising: (a) contacting a pool of ss nucleic acids with a metal donor so that an inorganic compound product comprising the metal is assembled; (b) partitioning nucleic acids that assemble inorganic compounds having a selected property; (c) generating an enriched pool of ss nucleic acids; and (d) repeating (a) to (c) at least one additional time to produce an inorganic compound product, thereby isolating a ss nucleic acid which is able to assemble an inorganic compound product. Optionally, the nucleotide sequence of the ss nucleic acid(s) is determined.

The invention further encompasses affinity purification methods using ss nucleic acids that bind to inorganic compounds. For example, a ss nucleic acid that selectively recognizes and binds to a particle having a particular crystal shape can be employed to isolate, identify and/or sort such crystals.

As another aspect, the present invention provides the inorganic compounds and ss nucleic acids discovered and/or synthesized using the methods of the invention. In one illustrative embodiment, the inorganic compound is a palladium plate (e.g., having a size of at least about 10, 25, 50, 100, 150 nM and/or less than about 50, 100, 150, 200, 250, 300 nM or more), optionally a magnetic or ferromagnetic palladium plate. The invention further encompasses novel cobalt-iron oxides, including cobalt-iron oxide spheres, cubes, fibers, nanocapsules and nanotubes (e.g., having a size of at least about 1, 5, 10, 20, 40, 50 or 60 nM and/or less than about 50, 75, 100, 150 or 200 nM or more in diameter), which can also be synthesized using the methods and ss nucleic acids of the invention.

The present invention has numerous applications for inorganic compound synthesis and discovery, including the synthesis and/or discovery of new materials, particle shapes and catalysts. For example, in particular embodiments, the invention is practiced to synthesize inorganic plates, nanotubes, nanocapsules, spheres, cubes or fibers/wires. In other embodiments, the inventive methods are practiced to identify catalysts (e.g., for alkene hydrogenation, alcohol oxidation, water splitting, methanol oxidation, etc.) or materials for hydrogen storage and sensing. In particular, current catalysts generally rely on expensive metals such as palladium and platinum. The present invention can be practiced to identify less expensive catalysts, e.g., those containing aluminum, nickel, iron, etc., or alloys or intermetallics thereof.

Further, the ss nucleic acids of the invention can be employed to discover and/or synthesize catalysts for use in direct methanol fuel cells. In particular embodiments, the catalyst comprises an alloy comprising a transition metal (e.g., palladium, platinum, ruthenium). Most current methanol oxidation catalysts are alloys of mid-transition row elements such as platinum, ruthenium, osmium, rhodium and iridium. Mallouk (Sun et al. (2001) Anal. Chem. 73: 1599-1604; Reddington et al., (1998) Science, 280: p. 1735-1737) described identification of a highly active quaternary alloy using a method in which catalysts of varying composition were printed from an ink-jet printer onto carbon electrodes. The most active catalyst was comprised of Pt(44)/Ru(40)/Os(10)/Ir(5) (numbers indicate atomic percentages). This work highlights the challenges associated with materials discovery. Material properties are substantially impacted by composition; narrow regions in the composition space often define superior materials that are difficult to identify using conventional processing techniques. In contrast, the methods of the present invention can be readily used to select among a large and diverse array of materials to identify those with a specified property. Further, the methods of the invention do not require pre-existing information to drive the selection process.

In another illustrative embodiment, the invention provides improved palladium plates for hydrogen storage and sensing. Hydrogen intercalates with palladium and can thereby be stored in palladium compositions. The discovery by the present inventors of novel two-dimensional palladium plates provides an improved storage medium due to the large surface area. With respect to hydrogen sensing, release of intercalated hydrogen produces a change in the conductivity of palladium compositions, which can be detected (e.g., by electrodes). The palladium materials of the present invention may be more sensitive to hydrogen release. Further, release of stored hydrogen may result in changes in the properties (e.g., magnetism) of the palladium materials of the invention, which can also be detected by methods known in the art.

As still another application, the present invention can be practiced to identify and/or synthesize materials for the manufacture of silicon quantum dots, photovoltaics (e.g., solar panels), and field emitters.

Other non-limiting uses of the present invention include synthesis of high tech materials, e.g., for use in the defense industry and synthesis of materials for the manufacture of transparent semi-conductors, magnetic semi-conductors, and superconductors.

Functional ss nucleic acids can also be identified according to the invention that distinguish among different inorganic crystal shapes. Thus, the invention can be used to sort or affinity purify a desired inorganic crystal structure.

The following Examples are provided to illustrate the present invention and should not be construed as limiting thereof.

### EXAMPLE 1

### Synthesis of Magnetic Plates

This Example describes the use of modified RNA libraries with enhanced metal binding affinity for inorganic particle formation. A modified RNA library was generated to select for RNA molecules with enhanced metal binding. The selection cycle used for discovering RNA-mediated crystal growth is shown in FIG. 1. The selection began with a chemically-synthesized (ABI 391) library of 10¹⁴ unique ssDNA sequences, 87 bp in length, containing a center region of 40 bp, random in sequence and flanking sequences which were specific for T7 RNA polymerase priming. Two cycle PCR was used to generate a dsDNA library. In step 1, T7 RNA polymerase was used to transcribe the dsDNA library into a ssRNA library containing ca. 10¹⁴ sequences. During step 1,5-(4-pyridylmethyl)-UTP (*UTP) was used to provide additional metal coordination sites beyond the heterocyclic nitrogens present in native RNA. As an alternative, a modified CTP analog could be used. In step 2, the RNA library (500 nM) was incubated in two parallel selection experiments with the metal complex dibenzylideneacetone palladium(0) ([Pd₂(DBA)₃]), at 100 μM or 400 μM to provide a source of Pd⁰ atoms (FIG. 1). The incubation was performed in aqueous solution for 2 hours at ambient temperature. For selection step 3 to be successful, RNA sequences either mediated the formation of Pd metal particles and remain bound to those particles, or simply bound to particles formed spontaneously or by other RNA sequences. Size exclusion membranes (Microcon™ 100, 100 kD cutoff) were used to select for particles that were formed in the presence of RNA. Initial partitioning rounds gave<0.5% RNA retained by the Microcon™ filter as determined by scintillation counting of ³²P-labeled RNA. For further purification, an alternative denaturing gel electrophoresis separation can be performed, wherein the RNA-Pd conjugates are isolated from gel slices. The selected RNA was reverse-transcribed (step 4, AMV reverse transcriptase) to give a cDNA copy of the desired RNA sequences. PCR amplification completed the selection cycle and provided a dsDNA template enriched in the desired sequences and ready for T7 RNA polymerase transcription and the beginning of the next cycle. Initially (cycles 1-3), partitioning (step 3) was accomplished by a 100 kD cutoff filter. In cycles 4 to 8 the 100 kD molecular weight cutoff filter was followed by native polyacrylamide gel (6%) electrophoresis gel-mobility shift-dependent partitioning. Slowly migrating bands, relative to the starting RNA transcript, showing a dependence on both RNA and Pd, were isolated. It was shown that Pd particle formation was not dependent on the Microcon™ filter.

It should be noted that if the partitioning of step 3 was 100% effective at separating the active from the inactive RNA sequences; this would be a one cycle technique. However, only a small fraction (ca. hundreds) of the starting 1014 RNA sequences were active, making it likely that a small amount of inactive RNA sequences were carried along in step 3. For this reason the cycle is desirably repeated several times.

As an alternative step in the isolation of RNA-Pd conjugates, a property-based selection can be performed, wherein only those RNA sequences capable of growing magnetic Pd particles are carried forward to subsequent cycles. The property-based selection (i.e., magnetic field selection) is carried out by placing a magnet under a vial containing the Pd plates. Magnetic Pd particles are attracted to and held in the bottom of the vial while any non-magnetic particles are washed away: The particles retained by the magnet (i.e., ferromagnetic or magnetic) are then isolated by non-denaturing polyacrylamide gel electrophoresis.

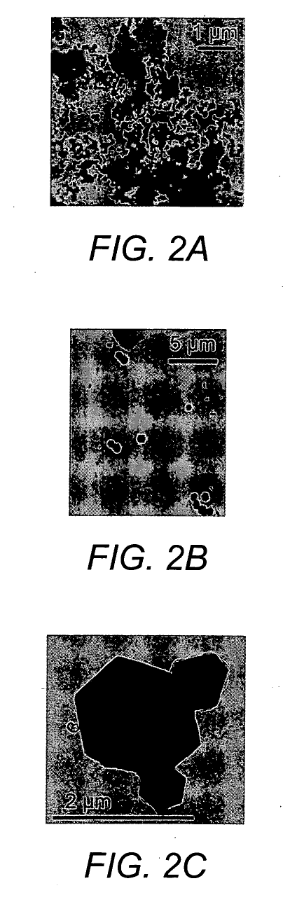

Transmission electron microscopy (TEM) analysis of the Pd particles produced in 2 hours by the starting random RNA library revealed mostly small (5 nm diameter) particles of undefined shape (FIG. 2A). TEM analysis of the Pd particles created by the evolved RNA cycle 8 pool after 2 hours were strikingly different. The dominant Pd particle shape observed was thin hexagonal plates (FIGS. 2B and 2C). Also observed at lower frequency (ca. 1%) were cubes and rods. A combination of scanning electron microscopy (SEM) and electron diffraction showed that the hexagonal particles were crystalline Pd. Further characterization by atomic force microscopy showed the Pd particles to be approximately 20 nm in thickness. Control experiments using polyvinylpyridine under identical incubation and isolation conditions gave no particle growth. From the analyses of the evolved RNA pool, it was unclear if a single RNA sequence was sufficient to create these particles, or if multiple RNA sequences in the pool were mediating particle growth.

To investigate individual sequences, the RNA pool was cloned and sequenced to yield individual RNA isolates. The isolates could be grouped into families, indicating that a RNA biopolymer can evolve in response to an inorganic materials synthesis pressure. Exemplary RNA sequences obtained are listed in Table 1 and are grouped in families based on conserved sequence regions. Completely conserved sequence regions are in uppercase letters. Underlined regions indicate some sequence relationships between families. Members of families 1 and 2 may be the result of mutations or deletions/insertions of individual sequences present in the starting pool. Families 3, 4 and the orphan sequence appear to be discrete isolates based on the relatively long regions of non-homologous sequence flanking the conserved regions. Further, family 4 sequences are related by their 5′-end conserved region and show sequence similarity to both families 1 and 2.

TABLE 1

| | | |
|---|---|---|
| | | SEQ ID |
| Isolate | Nucleic Acid Sequence | NO: |

Family 1 (14 members, 56%)

| | | |
|---|---|---|
| PD_017 | cccuuucuauccucaauguaccaacaAAAAAUGUA | 1 |
| | uucc | |
| PD_021 | cucuuccuauccucaaaguaccaacuAAAAAUGUA | 2 |
| | cgccc | |
| PD_024 | cccuuucuaucuucaauguaccaacuAAAAAUGUA | 3 |
| | uuccc | |
| PD_025 | cccuuucuauccucaauguaccaacuAAAAAUGUA | 4 |
| | uuccc | |
| PD_028 | cccuuccuauuuccaaugucccaacaAAAAAUGUA | 5 |
| | uuccc | |
| PD_029 | cccuuucuauccucaauguaccaacaAAAAAUGUA | 6 |
| | uuccc | |
| PD_031 | cccuuccuauuuccaaugucccaacaAAAAAUGUA | 7 |
| | uuccc | |
| PD_032 | cccuuccuauuuccaaugucccaacaAAAAAUGUA | 8 |
| | uuccc | |
| PD_082 | cccuuccuaucuccaaugucccaacaAAAAAUGUA | 9 |
| | uuccc | |
| PD_085 | cccuuucuauccucaauguaccaacuAAAAAUGUA | 10 |
| | ugccc | |
| PD_086 | cccuuucuauucucaauguaccaacuAAAAAUGUA | 11 |
| | uuccc | |
| PD_090 | cccuuccuaucuucaaugucccaacuAAAAAUGUA | 12 |
| | uuccc | |
| PD_093 | cccuuccuauccccaaugucccaacaAAAAAUGUA | 13 |
| | ucccc | |
| PD_094 | cccuuccuauuuccaaugucccaacaAAAAAUGUA | 14 |
| | uuccc | |

Family 2 (6 members, 24%)

| | | |
|---|---|---|
| PD_019 | CUCCUUAAUACCUcaaaauaccccaucuuuacgua | 15 |
| | cguua | |
| PD_022 | CUCCUUAAUACCUuuuaauaccccaucuuucguaa | 16 |
| | cguua | |
| PD_026 | CUCCUUAAUACCUuaaaauaccccaucuuuaugua | 17 |
| | acguua | |
| PD_027 | CUCCUUAAUACCUuauaauaccccaucuuuacgaa | 18 |
| | cguua | |
| PD_030 | CUCCUUAAUACCUuuuaauaccccaucuuucguaa | 19 |
| | cguua | |
| PD_092 | CUCCUUAAUACCUuuuaauaccccaucuuucguaa | 19 |
| | cguua | |

Family 3 (2 members, 8%)

| | | |
|---|---|---|
| PD_020 | cucuUUAUUUCCUUaaaauaccaaaucuuaaugaa | 20 |
| | uccc | |
| PD_091 | cucuUUAUUUCCUUuauaguacccccucuuauugu | 21 |
| | aucgcc | |

Family 4 (2 members, 8%)

| | | |
|---|---|---|
| PD_081 | CCCCUCAAUaccuuuuaaUACCccaucuuucguac | 22 |
| | gucua | |
| PD_089 | CCCCUUCAAUcuucaaugUACCaacuauaaaugaa | 23 |
| | cgccc | |
| Orphan | | |
| PD_084 | cccuuucuuuuuucaaaguacccccuauuauugua | 24 |
| | uuuca | |

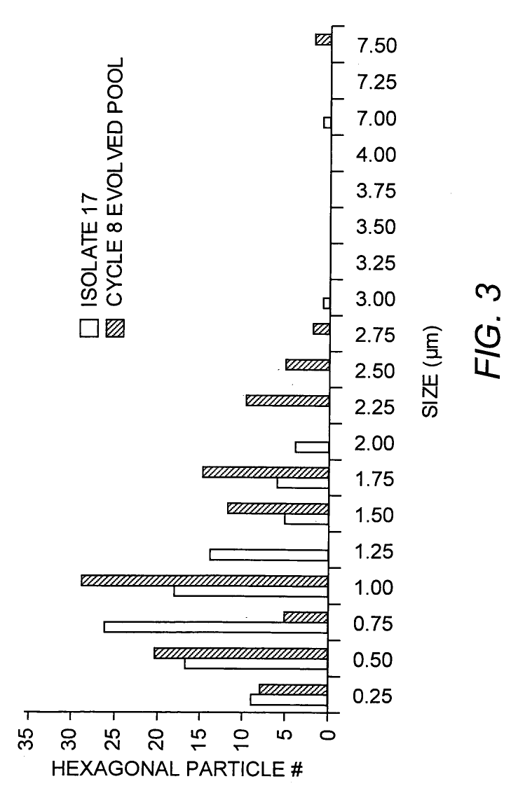

Isolates 17, 19, 20, 81, and 84 were chosen as representatives of the different families, and their ability to form particles was investigated by TEM. All isolates mediated the formation of hexagonal particles of similar structure to those shown in FIG. 2. Each of the RNA family representatives, in contrast to the cycle 8 pool, exclusively formed hexagonal particles. For this form of modified RNA and this selection procedure hexagonal plates were the dominant Pd particle form to evolve. Few methods exist for growing thin hexagonal Pd particles (Walter (2000) Adv. Mater. 12: 31-33) and the hexagonal Pd particles grown by these RNA isolates are distinctive in their large size and shape uniformity. FIG. 3 shows the distribution of Pd hexagonal particles measured by TEM for the evolved pool and isolate 17 after 2 hours incubation with Pd₂(DBA)₃ (100 μM). The average particle size was similar for both the evolved pool and isolate 17 (1.3±0.9 μm vs. 1.2±0.6 μm, respectively), however, the distribution of the particles was significantly narrower for isolate 17 (FIG. 3). This result indicates that although each family member directs the formation of the same final particle product, they do so at different rates.

Given that individual RNA isolates can mediate hexagonal Pd particle growth it was determined how fast the particles formed. For comparison, when synthetic polymers are used to direct crystal type and size, the concentration of polymer is typically in excess of inorganic precursor, and the reaction is performed at elevated temperature. Further, the concentration of the metal precursors is typically several orders of magnitude higher than that reported herein. The cycle 8 pool and isolate 17 were tested for their ability to mediate Pd particle growth at a range of times from 2 hours decreasing to 1 minute. Unexpectedly, 0.32 μm±0.27 μm wide hexagonal particles were formed by the RNA pool at 500 nM and Pd₂(DBA)₃ at 400 μM in 7.5 minutes. To determine if this rapid rate of particle growth required multiple sequences, isolate 17 was tested alone for its ability to mediate particle growth. Under identical conditions isolate 17 could grow Pd particles 1.3 μm±0.6 μm wide in 1 minute.

It has now been shown that RNA can mediate the formation of novel inorganic materials. The hexagonal Pd plates evolved over 8 cycles of in vitro selection cannot be easily produced by any other known methods. The presence of multiple RNA sequence families that mediate this novel particle growth indicates that this biopolymer can be an active participant in inorganic materials evolution.

### EXAMPLE 2

### Synthesis of Cobalt-Iron Oxide Particles

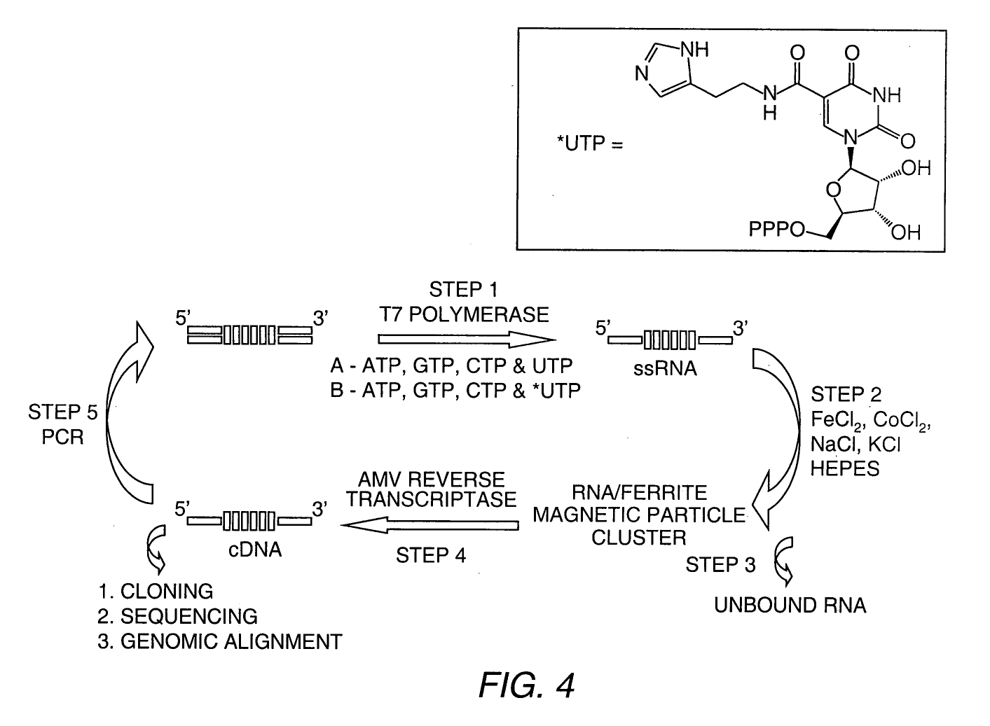

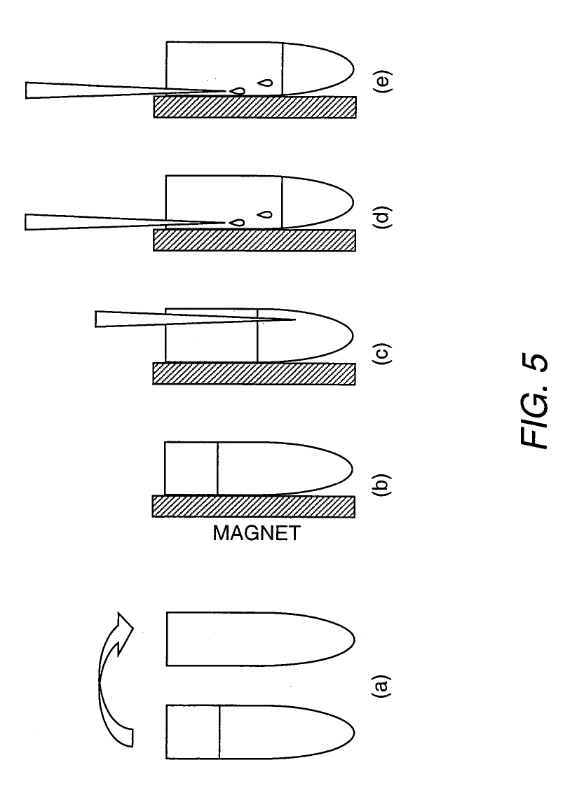

FIG. 4 shows the in vitro selection scheme used to synthesize and identify cobalt-iron oxide compounds having properties of interest, including cobalt-iron oxide spheres, cubes and fibers (including magnetic cobalt-iron oxides), as well as to identify functional RNA molecules involved in the formation of such cobalt-iron oxide compounds. A random ssDNA pool of 10¹⁴ molecules with different sequences was used in the selection cycles. The pool contained chemically-synthesized ssDNA (Invenex, Inc., Denver, Colo.) of 87-bp in length with a 40-bp long random region in the middle, flanked by defined sequences to allow for primer binding and enzymatic reactions applied in selection procedures. A pool of dsDNA, equivalent to the library of ssDNA pool, was generated by performing two cycles of PCR on the random ssDNA. In Step 1, two sets of complementary ssRNA pools were created by in vitro transcriptions. The ssRNA molecules were produced by incubating random dsDNA at 37° C. for 6 hours with T7 polymerase, T7 polymerase buffer, RNase inhibitor, ATP, CTP, GTP and UTP. Under similar conditions, a second pool of ssRNA was produced which had incorporated into the nucleic acid sequences an imidazole-modified UTP. The imidazole modification was introduced to act as an additional metal ligand to cobalt and iron. Radioactively labeled ATP [α-³²P] was used for RNA detection and quantitation. Transcripts were subsequently purified using a 10 K molecular weight cut-off filter, washed four times with 1× buffer (Na⁺, K⁺, PO₄²⁻) and resuspended in water. Radioactively labeled pure transcripts were quantitated by liquid scintillation counting. In Step 2, the RNA pool (450 pmol) was combined with cobalt-iron oxide precursors: 75 nmol FeCl₂ (75 μL, 1 mM solution), 37.5 nmol COCl₂ (37.5 μL, 1 mM solution) and 0.5 μmol KCl (20 μL, 2.5 M), 0.5 pmol NaCl (20 μL, 2.5 M), HEPES buffer (25 μL), deionized water (up to 500 μL) and incubated at room temperature for 5 hours. Following the incubation, magnetic nanoparticles containing RNA molecules were separated from remaining inactive RNA and unused reagents using magnet partitioning (FIG. 5); a tube containing incubated material was placed on a magnet for 12 hours. Upon removal of the solution, magnetic nanoparticles with bound RNA were attracted by the magnet and remained in the tube. The particles were washed four times with 200 μL of 1× buffer containing K⁺, Na⁺, and PO₄₃-ions to assure RNA stability thought the washing procedure. The particles were resuspended in 100 μL of deionized water and, in addition to the washes, were counted on a LS scintillation counter to monitor the active RNA recovery. To eliminate RNA molecules that bind to the sides of the tube a counter-selection step was introduced. Prior to magnetic partitioning the samples were transferred to a fresh tube so that any RNA bound to the tube was left behind.

In Step 4, partitioned RNA molecules were treated at 42° C. for 45 minutes and 72° C. for 15 minutes with SuperScript™ II RNase H⁻ Reverse Transcriptase, 3′-primer, dNTPs (dATP, dCTP, dGTP, dTTP), and 5×1^{st} strand buffer to generate a DNA copy of active RNA molecules. The cDNA copy of selected sequences was amplified, without purification, by means of PCR using 3′- and 5′-primers, dNTPs, 10× Taq DNA Polymerase buffer and Taq DNA Polymerase (8-16 cycles of 95° C. for 1 minute, 56° C. for 1 minute, and 75° C. for 30 seconds). The amplified DNA (of both active and inactive series) was purified using QIAquick PCR purification Kitm (QIAGEN®, Valencia, Calif.), and quantitated using either a pico green or an ethidium bromide assay. Pure DNA samples served as templates for T7 RNA Polymerase in the next cycle of selection.

In general, functional RNA molecules were not selected in a single selection cycle since they represent only a fraction of the starting pool. Inactive RNA tended to also be present, therefore the selection cycle is generally repeated multiple (e.g., 6-12) times. In this case, a population of RNA molecules directing the growth of cobalt-iron oxide magnetic particles was separated after eight rounds of the in vitro selection.

A series of controls were conducted in the partitioning step to demonstrate that RNA was being retained in the tube due to the binding to magnetic particles versus any nonspecific binding or aggregation. An additional set of incubations was prepared for each sample. The partitioning step for the control samples was conducted under similar conditions as those shown in FIG. 5, but without the presence of the magnet. Theoretically, no RNA should be retained in the control tubes because there was no magnet to prevent the removal of magnetic nanoparticles and RNA bound thereto. The amount of [a ³²P]-labeled RNA lost at each step of partitioning was monitored by liquid scintillation counting. In step a, all liquid was removed from the tubes, followed by four washes with 200 μL of 1× buffer (FIG. 5, steps b-e). The last step corresponded to resuspension of the material that remained in the tube, with 100 μL of deionized water. Control samples showed very low RNA retention in comparison to samples partitioned with the magnet.

To further demonstrate the significance of using a magnet in the partitioning step, a set of control incubations was prepared and partitioned without the magnet (as described above). Resuspended material was placed on copper TEM grids and investigated by transmission electron microscope. No colloid formation was detected.

Synthesis of CoFe₂O₄ colloids using imidazole monomer was also conducted with no RNA present. The imidazole control incubation was imaged under the TEM and no colloid formation was detected.

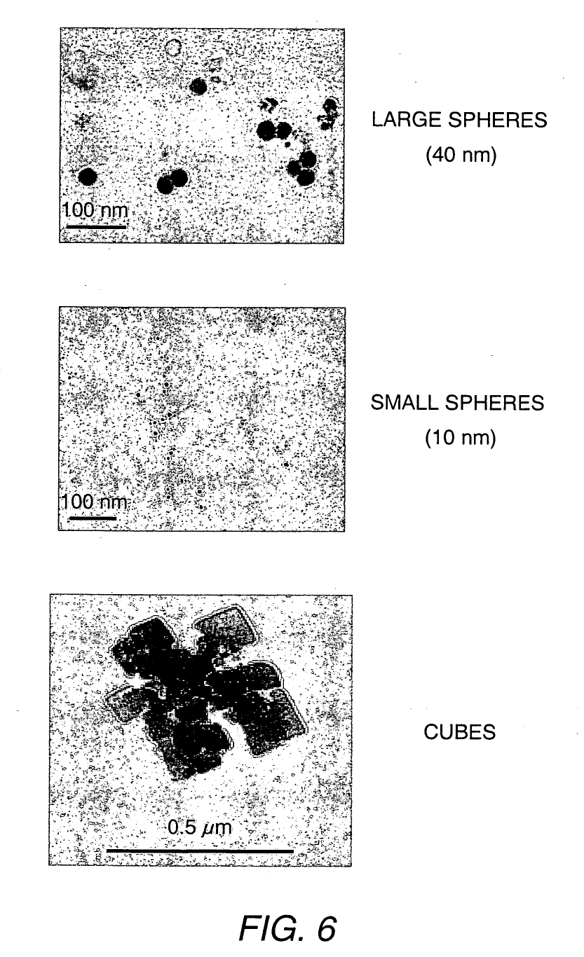

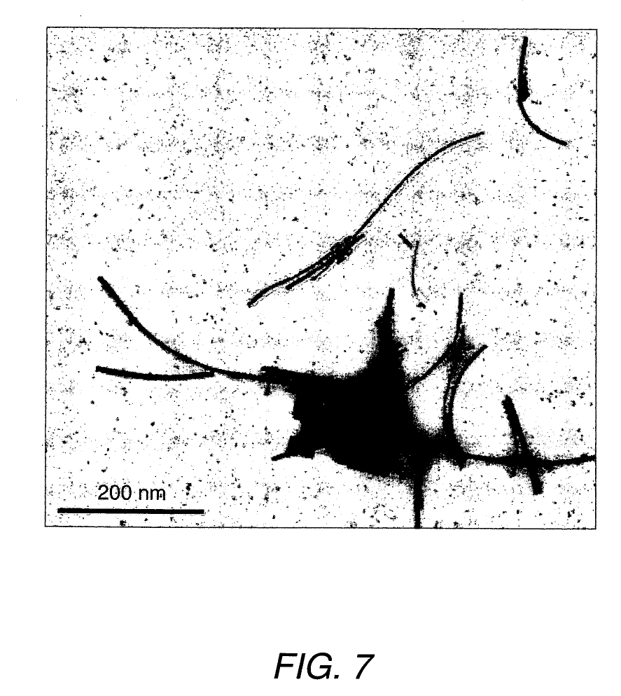

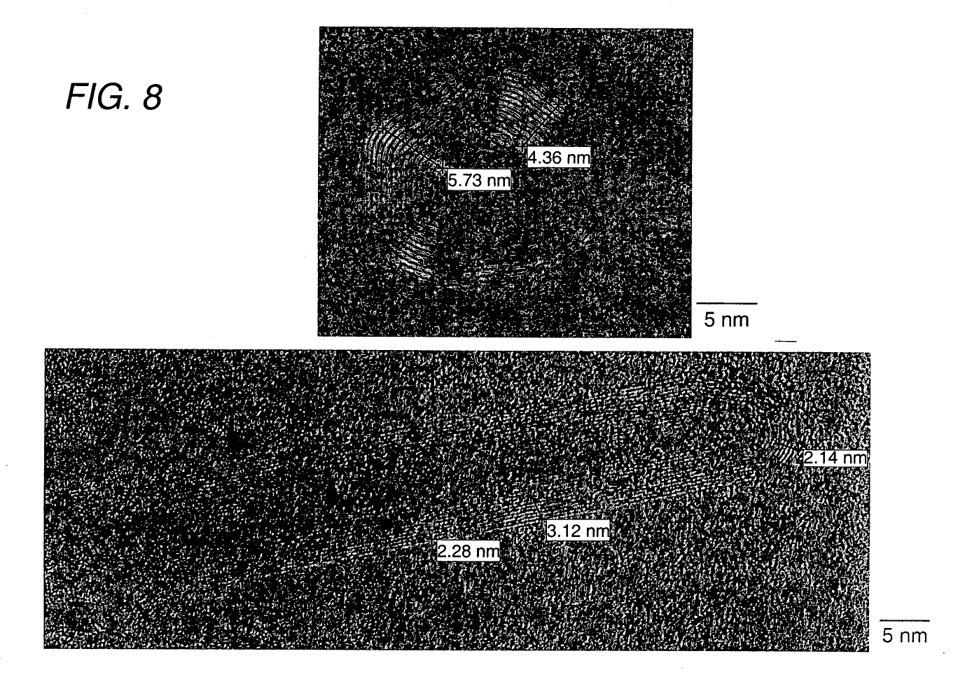

Analysis of particles containing active RNA molecules were analyzed and several different types of particles were retained by the magnet including large (˜40 nm) and small (˜10 nm) cobalt-iron oxide sphere and cubes (FIG. 6). The fraction that was not retained by the magnet contained large cobalt iron oxides in the form of fibers (FIG. 7). These particles are magnetic; they were not been retained by the magnet because the magnet was not large enough or was not applied for a long enough time to retain these larger particles. FIG. 8 shows electron micrograph images of magnetic cobalt iron oxide nanocapsules and nanotubes that were also formed.

To investigate individual sequences, the RNA pool was cloned and sequenced to yield individual RNA isolates. Exemplary RNA sequences obtained are listed in Table 2 and are grouped in patterns based on conserved sequence regions. Conserved sequence regions are in uppercase letters.

TABLE 2

| | | |
|---|---|---|
| | | SEQ |
| Isolate | Nucleic Acid Sequence | ID NO: |

| | |
|---|---|
| Pattern 1: TTTATTAA (10 members, 25%) | 25 |

| | | |
|---|---|---|
| Seq054 | acctattctcagccttcaTTTATTAAcagtccctac | 26 |
| | ttaa | |
| Seq2002 | agcttaataaacgcaacctcTTTATTAAttatctta | 27 |
| | gaca | |
| Seq2030 | cctcaTTTATTAAcaccaagttccttaactccctga | 28 |
| | atac | |
| Seq2049 | ccaacaattaaccttTTTATTAAtcaatcatatcct | 29 |
| | ttac | |
| Seq2070 | cctatatcaactcgtctttcatTTTATTAAcataat | 30 |
| | gtta | |
| Seq2010 | tcctttaactaattaccTTTATTCAActtacccaaa | 31 |
| | ata | |
| Seq2042 | cccctcacacatcttttcctagaTTTATTCAAccct | 32 |
| | acgt | |
| Seq2090 | cactttatttcacatttttgcccTTTTTTAAtctca | 33 |
| | cc | |
| Seq075 | aTTTCTTAAagccccaggcctttaacttaatccgtt | 34 |
| | catg | |
| Seq019 | tatacatgtctaatctgtgTTGATTAAtctattact | 35 |
| | c | |

| | |
|---|---|
| Pattern 2*: ACTACHAATAHGCTHHAATAAAAACAATWAC | 36 |
| WAWA (3 members, 7.5%) | |

| | | |
|---|---|---|
| Seq2005 | ggttATAATCAATATGCTCCAATAAAAATAAAAACT | 37 |
| | ATAc | |
| Seq2061 | CCTCCTAATGCACTATAATAAAAACAATTACAAAAg | 38 |
| Seq002 | ttctactatgaACTACATATAAGGTTAAATAAAATC | 39 |
| | TCT | |

| | |
|---|---|
| Pattern 3: TTTATTAACATNAHGTTMYT | 40 |
| (5 members, 12.5%) | |

| | | |
|---|---|---|
| Seq054 | acctattctcagccttcaTTTATTAACAGTCCCTAC | 26 |
| | TTaa | |
| Seq2030 | cctcaTTTATTAACACCAAGTTCCTTAactccctga | 28 |
| | atac | |
| Seq2070 | cctatatcaactcgtctttcatTTTATTAACATAAT | 30 |
| | GTTA | |
| Seq051 | cccatctcAATATTTACATCATGATACTatacttct | 41 |
| | tttc | |
| Seq2033 | cacttatctatttcataactagaatCCCATTAACAT | 42 |
| | GACC | |

Derivatives of Consensus Sequence Patterns AATAAAA
A (SEQ ID NO:43) and TTTATTAA (SEQ ID NO:25) (40
members, 92.5%)

| | | |
|---|---|---|
| Seq005 | tcgtcacacacacaatacaATTACTAAatcaagcca | 44 |
| | atca | |
| Seq019 | tatacatgtctaatctgtgTTGATTAAtctattact | 35 |
| | c | |
| Seq020 | aTGGTTTAAatttgaattccttgatctctcttttcc | 45 |
| | catc | |
| Seq051 | cccatctcAATATTTAcatcatgatactatacttct | 40 |
| | tttc | |
| Seq054 | acctattctcagccttcaTTTATTAAcagtccctac | 46 |
| | ttaa | |
| Seq055 | ttcctttaaactcttactctaagttatacaATTATA | 47 |
| | AT | |
| Seq065 | ccacacagttcctccctttggacctaAGAATTAAta | 48 |
| | ctta | |
| Seq075 | aTTTCTTAAagccccaggcctttaacttaatccgtt | 34 |
| | catg | |
| Seq2006 | actcacctccatattttacttgtctcgGTTGTTAAt | 49 |
| | ttag | |
| Seq2029 | ctcagattttttgTCTATTTAttgttttaactactt | 50 |
| | aact | |
| Seq2030 | cctcaTTTATTAAcaccaagttccttaactccctga | 51 |
| | atac | |
| Seq2034 | caacactacacTATATTCAcctttcattgcgcactc | 52 |
| | tcaa | |
| Seq2037 | TGTATTGCaccaacttactatatgtatatatttgta | 53 |
| | caca | |
| Seq2041 | acttagtcatcctaactccatctataTTTCTCAAa | 54 |
| Seq2042 | cccctcacacatcttttcctagaTTTATTCAaccct | 32 |
| | acgt | |
| Seq2050 | tatgcctccttctatattgtcgcgttatTTTATCCA | 55 |
| | cccc | |
| Seq2066 | tatgtgttgtagcgtcaatcaccgaatatgggaTAC | 56 |
| | ATTA | |
| Seq2069 | gatttccttatctcacacTTTTTTAGagactcctag | 57 |
| | caac | |
| Seq2073 | cGATATTTAattctaacctgcaaaccagccaacatc | 58 |
| | gcac | |
| Seq2074 | tagacTTTTCTATacccccatatatcttttttctct | 59 |
| | cata | |
| Seq2078 | tagcagGTTATATAcaaatgtcgaccttatagcttt | 60 |
| | ttct | |
| Seq2090 | cactttatttcacatttttgcccTTTTTTAAtctca | 33 |
| | cc | |
| Seq002 | ttctACTATGAActacatataaggTTAAATAAaatc | 39 |
| | tct | |
| Seq025 | cCATAAGAGtactctTGTAGTAActtcacaatttaa | 61 |
| | cttg | |
| Seq2002 | agcttAATAAACGcaacctcTTTATTAAttatctta | 27 |
| | gaca | |
| Seq2005 | gGTTATAATcaatatgctccAATAAAAAtaaaaact | 37 |
| | atac | |
| Seq2010 | tcctttaACTAATTAccTTTATTCAacttacccaaa | 31 |
| | ata | |
| Seq2017 | acacaattcccacAATCAAATtttaaaacatCCTAT | 62 |
| | TCA | |
| Seq2021 | ccgacactCTTATTCCtttccacactcGATAAAGTa | 63 |
| | catc | |
| Seq2026 | actccTCTATAACcacacattaaagttaaatcACCA | 64 |
| | AAAT | |
| Seq2033 | cacttatctaTTTCATAActagaatccCATTAACAt | 41 |
| | gacc | |
| Seq2045 | tatagacctactgcattagagttCATAATATgTCTC | 65 |
| | TTAT | |
| Seq2046 | TATCACAAaccTATCTTAAttccttatccttttgtc | 66 |
| | cctt | |
| Seq2049 | ccAACAATTAaccttTTTATTAAtcaatcatatcct | 29 |
| | ttac | |
| Seq2053 | ACTAATAAgtcatttctgtTATCTTAAtaaatttac | 67 |
| | gacg | |
| Seq2054 | tatctctaTCTTTTAGcctataagcACCAAAAAact | 68 |
| | tcct | |
| Seq2057 | tAATCATACtatattttgaatattggaacGTTATTA | 69 |
| Seq2061 | cctcctaatgcactatAATAAAAAcaATTACAAAag | 38 |
| Seq2070 | ccTATATCAActcgtctttcatTTTATTAAcataat | 30 |
| | gtta | |
| Seq2086 | taTTCAATAAcacttagagaccaccagtatcgCATA | 70 |
| | CAAA | |

| | |
|---|---|
| Derivatives of Pattern 2: AATAAAAA (2 | 43 |
| members, 5.0%) | |

| | | |
|---|---|---|
| Seq2038 | ATAAACCtcgtctaactcatacttacacaactaata | 71 |
| | cct | |
| Seq2058 | caacaCCTAAAAAatatatcgcctcatatacttgtg | 72 |
| | catc | |

Orphans (1 member, 2.5%)

| | | |
|---|---|---|
| Seq029 | tacataccctcatcagactttacatctttcacttcc | 73 |
| | ttct | |

*H = A, C, T
Y = C or T
M = A or C
W = A or T

### EXAMPLE 3

### Methanol Oxidation Cell

The invention can be practiced to synthesize a methanol oxidation catalyst and to identify ss nucleic acids that can form a methanol oxidation catalyst. Materials for oxidizing methanol at low over-potentials are of interest for direct methanol fuel cells. Methanol oxidation is kinetically slow because it requires the removal of 6 electrons in the overall reaction
CH₃OH+H₂O→6H⁺+CO₂+6e⁻ (1)

The best known methanol oxidation catalysts are alloys of mid-transition row elements such as Pt, Ru, Os, Rh, and Ir. A highly active quarternary alloy was discovered recently by Mallouk (Sun et al. (2001) Anal. Chem. 73: 1599-1604; Reddington et al., (1998) Science, 280: p. 1735-1737) using a method in which catalysts of varying composition were printed from an ink-jet printer onto carbon electrodes. To find this material, a library of over 600 compositions was screened simultaneously. The most active catalyst was comprised of Pt(44)/Ru(40)/Os(10)/Ir(5) (numbers are atomic percentages).

The in vitro RNA selection methods of the invention can be applied to combinatorial methanol oxidation catalyst discovery. In one particular embodiment, this is accomplished by:
(i) reducing transition metal precursors (Pd₂ DBA₃, K₂PtCl₄, RuCl₃) in the presence of a random sequence RNA pool to generate alloy particles,
(ii) evaporating the resulting sol onto a gold microelectrode,
(iii) stepping the electrode potential to a large positive potential (e.g., 0.9V vs. SCE) in an aqueous solution containing methanol, NaClO₄, and HClO₄ (pH 5),
(iv) collecting the RNA from the most active methanol oxidation catalysts, and
(v) repeating (i)-(iv) in successive selection rounds, with the additional selection constraint that the catalyst must oxidize methanol at lower applied potentials (step iii) in each round.

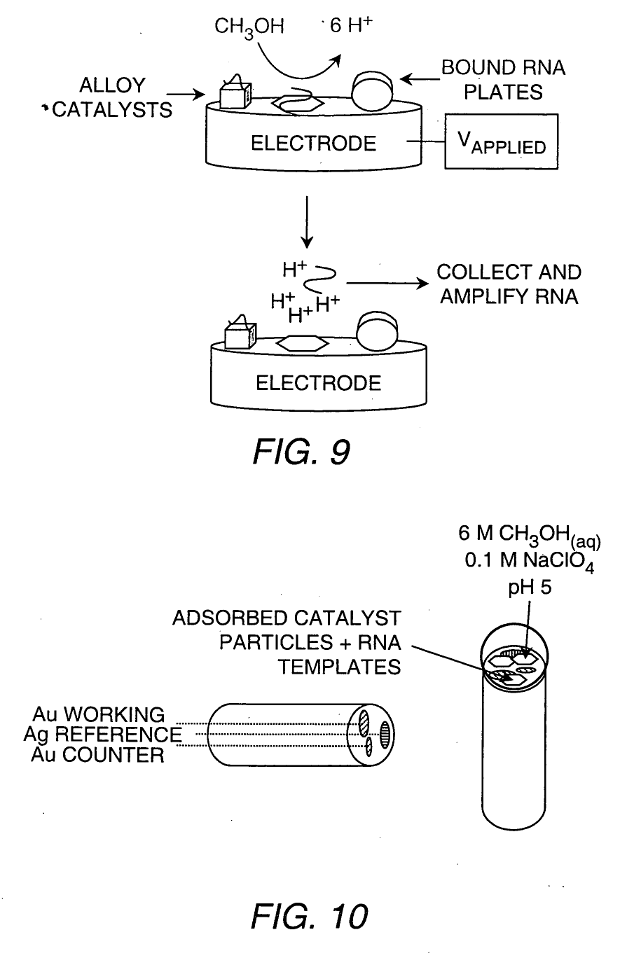

This general strategy capitalizes on the fact that RNA denatures at low pH. If a particular RNA sequence synthesizes an active catalyst, the local pH around that particle decreases as methanol is converted to protons (see equation 1). The RNA bound to that particle desorbs in response to the pH change and is collected and amplified in the next round (FIG. 9). The selection pressure can be more stringent as the number of selection cycles increases. For example, it is desirable to isolate RNA(s) that catalyze methanol oxidation at low applied potential. Early rounds of selection can be carried out at a relatively high applied potential, with lower applied potentials being used for selection during later rounds.

In the foregoing strategy, the particles physisorb strongly onto the electrode surface so that electrochemistry can be performed on them, and the active RNA sequences can be collected relatively easily. To simplify the experiment, the electrode configuration shown in FIG. 10 can be employed. The electrode houses three independently addressable microelectrodes, gold working and counter electrodes, and a silver reference electrode. Following evaporation of catalyst particles onto the electrode, the electrode is inverted and a drop (ca. 0.5 mL) of aqueous methanol solution is placed on top. An oxidizing potential is applied to the working electrode and after some time the methanol drop is drawn into a pipette and the RNA collected for reverse transcription and PCR. If the particles adsorb too weakly to the electrode surface such that they desorb when placed into the aqueous methanol solution, hexanedithiol can be assembled onto the gold electrode to covalently anchor the particles to the surface.

These methods can be employed using the Pd-RNA conjugates shown above. Alternatively, other metals as well as binary, ternary, and quaternary alloys can be used.

The foregoing is illustrative of the present invention, and is not to be construed as limiting thereof. The invention is defined by the following claims, with equivalents of the claims to be included therein.

1. A method of producing an inorganic compound product comprising contacting a single-stranded nucleic acid with a metal donor for a time and under conditions sufficient for the production of an inorganic compound product comprising the metal.

2. The method of claim 1, wherein the inorganic compound product has a size of from about 1 nm to about 20 μm.

3. The method of claim 1, wherein the single-stranded nucleic acid comprises RNA.

4. The method of claim 1, wherein the single-stranded nucleic acid comprises DNA.

5. The method of claim 1, wherein the single-stranded nucleic acid comprises a modified base.

6. The method of claim 5, wherein the single-stranded nucleic acid comprises a 2′-modified purine or pyrimidine base, a 5-position modified purine base, a 7-position modified pyrimidine base, or a combination thereof.

7. The method of claim 5, wherein the single-stranded nucleic acid comprises a thiol-modified uracil, a fluoro-modified uracil, a methoxy-modified uracil, an azido-modified uracil, an imidazole-modified uracil, a pyridyl-modified uracil, pyridylmethyl-modified uracil, an oxime-modified uracil, a carboxylate-modified uracil, an amine-modified uracil, a phosphine-modified uracil and/or a phosphite-modified uracil, or a combination thereof.

8. The method of claim 1, wherein the metal comprises at least one element selected from the group consisting of palladium, cobalt, platinum, silicon, aluminum, iron, rughenium, rhodium, osmium, iridium, copper and nickel.

9. The method of claim 1, wherein the inorganic compound product comprises an alloy.

10. The method of claim 1, wherein the inorganic compound product comprises an intermetallic compound.

11. The method of claim 1, wherein the inorganic compound product is a solid-state particle.

12. The method of claim 1, wherein the inorganic compound product is a soluble complex or colloid.

13. The method of claim 1, wherein the single-stranded nucleic acid is identified by a process comprising:
(a) contacting a pool of single-stranded nucleic acids with a metal donor so that an inorganic compound product comprising the metal is assembled;
(b) partitioning nucleic acids that assemble inorganic compounds having a selected property;
(c) generating an enriched pool of single-stranded nucleic acids from the partitioned single-stranded nucleic acids of (b); and
(d) repeating (a) to (c) at least one additional time to produce an inorganic compound product.

14. An inorganic compound product produced by the method of claim 1.

15. The inorganic compound product of claim 14, wherein the inorganic compound product has a size of from about 1 nm to about 20 μm.

16. The inorganic compound product of claim 14, wherein the inorganic compound product comprises at least one element selected from the group consisting of palladium, cobalt, platinum, silicon, aluminum, iron, rughenium, rhodium, osmium, iridium, copper and nickel.

17. The inorganic compound product of claim 14, wherein the inorganic compound product comprises an alloy.

18. The inorganic compound product of claim 14, wherein the inorganic compound product comprises an intermetallic compound.

19. The inorganic compound product of claim 14, wherein the inorganic compound product is a solid-state particle.

20. The inorganic compound product of claim 14, wherein the inorganic compound product is in the form of a plate.

21. The inorganic compound product of claim 20, wherein the plate comprises palladium or platinum.

22. The inorganic compound product of claim 14, wherein the inorganic compound product comprises cobalt-iron oxides.

23. The inorganic compound product of claim 14, wherein the inorganic compound product is in the form of a fiber.

24. The inorganic compound product of claim 23, wherein the fiber comprises cobalt-iron oxides.

25. The inorganic compound product of claim 14, wherein the inorganic compound product is in the form of a nanotube.

26. The inorganic compound product of claim 25, wherein the nanotube comprises cobalt-iron oxides.

27. The inorganic compound product of claim 14, wherein the inorganic compound product is a soluble complex or colloid.

28. A method of producing an inorganic compound product comprising:
(a) contacting a pool of single-stranded nucleic acids with a metal donor so that an inorganic compound product comprising the metal is assembled;
(b) partitioning nucleic acids that assemble inorganic compound products having a selected property;
(c) generating an enriched pool of single-stranded nucleic acids from the partitioned single-stranded nucleic acids of (b); and
(d) repeating (a) to (c) at least one additional time to produce an inorganic compound product.

29. The method of claim 28, wherein the inorganic compound product has a size of from about 1 nm to about 20 μm.

30. The method of claim 28, wherein the initial pool comprises from about 10⁸ to about 10¹⁷ independent single-stranded nucleic acid sequences.

31. The method of claim 28, wherein the single-stranded nucleic acids are RNA molecules.

32. The method of claim 28, wherein the single-stranded nucleic acids are DNA molecules.

33. The method of claim 28, wherein the single-stranded nucleic acids comprise a modified base.

34. The method of claim 33, wherein the single-stranded nucleic acid comprises a 2′-position modified purine or pyrimidine base, a 5-position modified purine base, a 7-position modified pyrimidine base, or a combination thereof.

35. The method of claim 33, wherein the single-stranded nucleic acid comprises a thiol-modified uracil, a fluoro-modified uracil, a methoxy-modified uracil, an azido-modified uracil, an imidazole-modified uracil, a pyridyl-modified uracil, pyridylmethyl-modified uracil, an oxime-modified uracil, a carboxylate-modified uracil, an amine-modified uracil, a phosphine-modified uracil and/or a phosphite-modified uracil, or a combination thereof.

36. The method of claim 28, wherein the metal comprises at least one element selected from the group consisting of palladium, cobalt, platinum, silicon, aluminum, iron, rughenium, rhodium, osmium, iridium, copper and nickel.

37. The method of claim 28, wherein the inorganic compound product comprises an alloy.

38. The method of claim 28, wherein the inorganic compound product comprises an intermetallic compound.

39. The method of claim 28, wherein the selected property is selected from the group consisting of size, a magnetic property, shape, an optical property, luminescence, fluorescence, an electronic property, photophysical property, crystal structure and a catalytic property.

40. The method of claim 39, wherein the selected property is size and the partitioning is carried out electrophoretically or magnetically.

41. The method of claim 39, wherein the selected property is a magnetic property and the partitioning is carried out magnetically.

42. The method of claim 28, wherein (a) to (c) are repeated at least five times.

43. The method of claim 42, wherein increasing selection pressure is applied over the course of successive iterations of (a) to (c).

44. The method of claim 42, wherein two or more selection criteria are applied in partitioning the nucleic acids.

45. The method of claim 28, wherein generating an enriched pool comprises a nucleic acid amplification.

46. A method of isolating a single-stranded nucleic acid which is able to assemble an inorganic compound product, comprising:
(a) contacting a pool of single-stranded nucleic acids with a metal donor so that an inorganic compound product comprising the metal is assembled;
(b) partitioning nucleic acids that assemble inorganic compounds having a selected property;
(c) generating an enriched pool of single-stranded nucleic acids; and
(d) repeating (a) to (c) at least one additional time to produce an inorganic compound product, thereby isolating a single-stranded nucleic acid which is able to assemble an inorganic compound product.

47. The method of claim 46, wherein the inorganic compound product has a size of from about 1 nm to about 20 μm.

48. The method of claim 46, further comprising determining the sequence of the single-stranded nucleic acid(s) in the enriched library.

49. The method of claim 46, wherein the initial pool comprises from about 10⁸ to about 10¹⁷ independent single-stranded nucleic acid sequences.

50. The method of claim 46, wherein the single-stranded nucleic acids are RNA molecules.

51. The method of claim 46, wherein the single-stranded nucleic acids are DNA molecules.

52. The method of claim 46, wherein the single-stranded nucleic acids comprise a modified base.

53. The method of claim 52, wherein the single-stranded nucleic acid comprises a 2′-position modified purine or pyrimidine base, a 5-position modified purine base, a 7-position modified pyrimidine base, or a combination thereof.

54. The method of claim 52, wherein the single-stranded nucleic acid comprises a thiol-modified uracil, a fluoro-modified uracil, a methoxy-modified uracil, an azido-modified uracil, an imidazole-modified uracil, a pyridyl-modified uracil, pyridylmethyl-modified uracil, an oxime-modified uracil, a carboxylate-modified uracil, an amine-modified uracil, a phosphine-modified uracil and/or a phosphite-modified uracil, or a combination thereof.

55. The method of claim 46, wherein (a) to (c) are repeated at least five times.

56. The method of claim 46, wherein generating an enriched pool comprises a nucleic acid amplification.

57. A method of producing an inorganic compound product comprising:
(a) contacting a pool of single-stranded RNAs with a metal donor so that an inorganic compound product comprising the metal and having a size of from about 1 nm to about 20 μm is assembled;
(b) partitioning single-stranded RNAs that assemble inorganic compound products having a selected property;
(c) amplifying the partitioned single-stranded RNAs of (b) to generate an enriched pool of single-stranded RNAs; and
(d) repeating (a) to (c) at least one additional time to produce an inorganic compound product.

58. An inorganic solid-state material consisting essentially of a palladium plate and having a size of at least about 50 nanometers.

59. The inorganic solid-state material of claim 58, wherein the palladium plate is ferromagnetic.

60. An inorganic solid-state material consisting essentially of a cobalt-iron oxide fiber.

61. An inorganic solid-state material consisting essentially of a cobalt-iron oxide nanotube.
